(19) United States
(12) Patent Application Publication
(10) Pub. No.: US 2009/0305258 A1
(43) Pub. Date: Dec. 10, 2009

(54) METHODS FOR THE DIAGNOSIS OF PROLIFERATIVE AND/OR CONFORMATIONAL DISEASES

(75) Inventors: Paolo La Colla, Cagliari (IT); Alessandra Pani, Cagliari (IT); Sandra Dessí, Cagliari (IT)

Correspondence Address: Kenneth K. Sharples;THE LAW OFFICE OF KENNETH K. SHARPLES, Sena Plaza Building, 125 East Place Avenue Suite 54, Santa Fe, NM, 87501, US

(73) Assignees: PAOLO LA COLLA, CAGLIARI (IT); ALESSANDRA PANI, CAGLIARI (IT); SANDRA DESSI', CAGLIARI (IT)

(21) Appl. No.: 12/279,715

(22) PCT Filed: Feb. 19, 2007
(86) PCT No.: PCT/IT07/00110

§ 371 Date: May 19, 2009

Related U.S. Application Data
(60) Provisional application No. 60/774,311, filed on Feb. 17, 2006.

(30) Foreign Application Priority Data
May 29, 2006 (IT) RM2006 A 000285

(51) Int. Cl. C12Q 1/68; C12Q 1/60

(52) U.S. Cl. 435/6; 435/11

(57) ABSTRACT

The present invention discloses methods to diagnose and/or to make prognostic predictions and/or to monitor the efficacy of a therapy of a proliferative or conformational disease, or to establish the state of ageing in a subject. Kit for performing such methods are also disclosed.

### FIELD OF THE INVENTION

This invention relates to studies on cause-effect relationships between alterations of cholesterol homeostasis, the ageing process and the development of proliferative diseases (such as atherosclerosis and neoplasms) and/or conformational diseases (such as Alzheimer's disease (AD) and prion-related diseases) in humans and/or other mammals.

This invention describes methods allowing to distinguish healthy subjects from subjects affected by, or at risk of developing, the above mentioned proliferative and/or conformational diseases.

More specifically, the present invention provides methods for assessing cholesterol trafficking and metabolism in peripheral cells, such as peripheral blood mononuclear cells (PBMCs) or skin fibroblasts. The methods encompass in vitro assays aimed at determining the levels of the following parameters:

i) total cholesterol, HDL- and LDL-cholesterol in serum, which can be used for differential diagnosis;
ii) free cholesterol (FC) in plasma membranes and esterified cholesterol (EC) in the cell cytoplasm;
iii) proteins (SREBP2, LDL-R, HMG-CoA-R, MDR1-Pgp, ACAT, nCEH, caveolin-1 and ABCA-1) and related mRNAs involved in the intracellular trafficking and metabolism of cholesterol;
iv) proteins (APP, Neprilysin, β-secretase, PrP, tumor suppressor proteins, oncoproteins) and related mRNAs involved in the pathogenesis of specific proliferative and/or conformational diseases;
v) pro-inflammatory cytokines (TNFα, IL-1α, IFNγ) and related mRNAs.

### BACKGROUND OF THE INVENTION

### Cholesterol Trafficking and Metabolism in Peripheral Cells

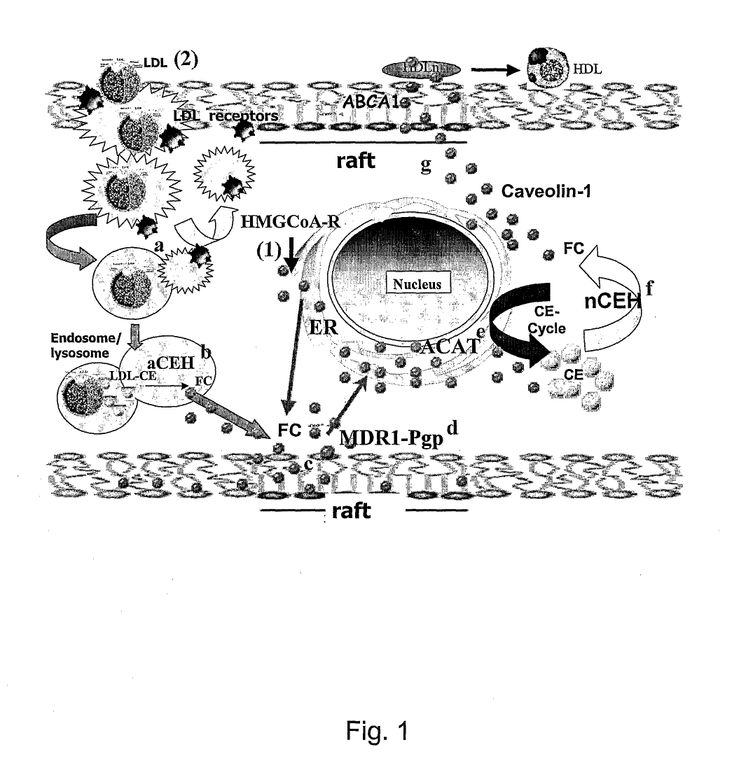

As summarized in FIG. 1, intracellular cholesterol derives from: i) endogenous neosynthesis (1) in the ergastoplasmic reticulum (ER) through the activity of hydroxyl-methyl-glutaryl-coenzime-A reductase (HMGCoA-R) and ii) circulating low density lipoproteins (LDL) (2), which are first internalised via LDL receptors (a) and then hydrolytically processed in lysosomes to generate free cholesterol (FC) through the activity of acid cholesterol ester hydrolase (aCEH) (b).

Most of the newly synthesized FC participates to the physiological turnover of cholesterol in the rafts (c), and/or to the biogenesis of new membrane domains in ER and Golgi.

If plasma membrane FC exceeds a threshold level, the excess FC is rapidly transported to the ER (d) by a P-glycoprotein (MDR1-Pgp, firstly described for its ability to catalyse ATP-dependent efflux of cytotoxic agents from tumour cells) (1-3), encoded by the multidrug resistance (MDR1) gene. Then, the ER-resident enzyme acyl-coenzyme-A cholesterol-acyl-transferase (ACAT) (e) converts FC into cholesteryl-esters (CE), which are stored in droplets in the cytoplasm (4-6). Although neosynthesized FC in the ER could be efficiently used for esterification by virtue of its proximity to ACAT, only a small percentage of it is directly esterified. Virtually all the esterified FC derives from the plasma membrane.

If cells require cholesterol, CE is re-hydrolyzed to FC by the enzyme neutral cholesterol-esters-hydrolase (nCEH) (f) and re-delivered to the plasma membrane by caveolin-1. If in excess, FC is eliminated from the cells through an efflux pathway spanning from the ER to the plasma membrane and involving caveolin-1, the ATP-Binding Cassette of the sub-family A, member 1 (ABC-A1), and plasma HDLs (g) (4-9).

### Role of Cholesterol in Lipid Rafts

Recent work has led to a new way of considering biological membranes, which are now viewed as a “mosaic of lipid domains”, rather than as an “homogeneous fluid mosaic”. It appears that cholesterol is not uniformly distributed within membranes, but is distributed into cholesterol-poor and cholesterol-rich domains. Among the latter, those containing saturated sphingolipids are referred to as lipid rafts (10), which float freely in plasma membranes carrying a few passenger proteins. When the passenger proteins are activated by a ligand, lipid rafts coalesce to form larger platforms where many different proteins converge in order to perform specific functions, such as signalling, processing or transport (10, 11).

Examples of raft passenger proteins are receptors for growth factors, signal transducing proteins (P21Ras), chemokine receptors, proteins of the MHC classes, antigen receptors, and various proteins with yet undefined functions, such as the amyloid precursor protein (APP) and the cellular prion protein (PrPc) involved in AD and Prion-related disorders, respectively. Interestingly, the amount of cholesterol associated with these domains exerts profound effects on the functions of the raft-resident proteins. For instance, perduring low levels of cholesterol in lipid rafts lead to a continuous stimulus for cell growth promotion (12), or induce APP and cellular prion protein PrPc to undergo pathologic processes leading to the generation of their corresponding pathogenic forms: the amyloidogenic A-beta peptide and the scrapie prion protein (PrPsc), respectively (13, 14).

### Cholesterol Trafficking and Metabolism in Proliferative Diseases

The initial studies of the authors on cholesterol trafficking and metabolism in proliferative diseases have been carried out in: i), rat acinar cell pancreatic carcinoma and ascite hepatoma (15, 16); ii) tissue biopsies from human solid and haematologic neoplasms (17-20); iii) cell lines derived from various human and animal tumours (21-23) and, iv) vessel tissues from healthy and atherosclerotic patients (24, 25).

In these in vivo and in vitro models the authors have demonstrated that the rate of cell proliferation correlates: i) positively, with the levels of MDR1-Pgp and ACAT and related mRNAs, leading to FC esterification; and ii) negatively, with the levels of nCEH and caveolin-1 and relative mRNAs, leading to intracellular CE accumulation (Table 1).

Accordingly, when compared to healthy controls, subjects affected by the above mentioned proliferative diseases possess lower FC levels, significantly higher CE levels and lower HDL-cholesterol levels.

TABLE 1
Correlations of intracellular free (FC) and esterified (CE) cholesterol
with plasma HDL-cholesterol in proliferative diseases.

| | | | HDL- | |
|---|---|---|---|---|
| Proliferative Diseases | FC | CE | Cholesterol | References |
| Rat ascite hepatoma | μg/10⁷ cells | | mg/dl | |
| 4 days after | 39.9 | 9.0 | 29 | 16 |
| transplantation | | | | |
| 7 days after | 35.6 | 18.0 | 27 | |
| transplantation | | | | |
| Controls | | | 42 | |
| Human neoplasias | mg/g tissue | | mg/dl | |
| Adult lung cancer | 1.0 | 1.0 | 15-35 | 18 |
| Controls | 1.4 | 0.25 | 45-60 | |
| Adult gastrointest. | 1.8 | 0.8 | 12-25 | 19 |
| Cancer | | | | |
| Controls | 1.5 | 0.2 | 45-60 | |
| Child solid cancers | | | 20-30 | 20 |
| Controls | | | 40-45 | |
| During remission | | | 40-45 | |
| | μg/mg protein | | mg/dl | |
| Adult haemat. neoplasias | | | 10-25 | 17 |
| Controls | | | 40-50 | |
| Child haemat. Neoplasias | | | 15-25 | 20 |
| Child solid cancers | | | 20-30 | |
| Leukemia cell lines | μg/mg protein | | mg/dl | |
| CEM (DT = 18 h) | 13.5 | 16 | Not | 22 |
| | | | Applicable | |
| MOLT-4 (DT = 25 h) | 17.5 | 4.0 | Not | |
| | | | Applicable | |
| L1210 (DT = 12 h) | | 22 | Not | |
| | | | Applicable | |
| Atherosclerosis | mg/g tissue | | mg/dl | |
| atherosclerotic plaque | | 9.0 | 18-30 | 44, 25, 24 |
| adjacent artery segments | | 4.0 | 18-30 | |
| arteriovenous fistulae | | 6.5 | | |
| healthy prone arteries | | 1.8 | 45-50 | |
| healthy resistant arteries | | 1.0 | 45-50 | |
| Veins | | 1.1 | 45-50 | |

However such an experimental approach, based on the analysis of tissue samples, is not readily applicable to the elaboration of an easy and fast test for the diagnosis and/or prognosis of proliferative or conformational diseases. The authors disclose in the present invention that PBMCs and/or fibroblasts, preferably PBMCs, are good starting materials to perform assays aimed at the identification of mRNAs and/or its protein products involved in intracellular cholesterol homeostasis.

### Cholesterol Trafficking and Metabolism in Conformational Diseases

Among the increasing number of neurodegenerative pathologies classified as conformational diseases, AD and Prion-related disorders (also known as Transmissible Spongiform Encephalopaties, TSEs) can be considered prototypes of non-transmissible and transmissible cerebral amyloidoses, respectively (25-27).

Although there are obvious differences in the aetiology and pathogenesis of these diseases, intracellular CE accumulation is a common hallmark indicating a link between these pathogenetic processes and the alteration of cholesterol homeostasis.

### Unsolved Problems

All the above diseases have reached serious proportions in terms of incidence. However, no conclusive means of diagnosis, therapy or prevention are available yet for AD and prion-related disorders (28-30).

### DESCRIPTION OF THE INVENTION

The present invention:

a) is useful to identify a cell phenotype possibly predisposing to pathological conditions;
b) contributes to the early diagnosis of suspected AD and TSE by providing a sensitive test before signs and symptoms become fully apparent;
c) represents a tool to assess the risk of developing AD among relatives of AD patients;
d) provides an easy method to study several cellular and plasma parameters involved in cholesterol metabolism, which are altered in various conformational and proliferative diseases, and then serves as an indicator of the effectiveness of therapy.

This invention allows to distinguish between clinically normal individuals and individuals affected by, or at risk to develop, proliferative and/or conformational diseases characterized by alterations in CE metabolism, which influences raft-associated protein function. This invention provides means for the diagnosis, prophylaxis, therapy and therapy monitoring of proliferative and/or conformational diseases.

In addition, it provides means for predicting/establishing the therapeutic/prophylactic value of existing or of new, synthetic or natural compounds/active principles for the ageing process, proliferative or conformational diseases.

In any event, when used either alone or in conjunction with other tests (i.e. ApoE aplotype), the methods of the present invention will improve the probability of correctly diagnosing the presence, the risk, or the absence of proliferative diseases, conformational diseases, such as AD, or prion-related diseases, such as TSE.

The present invention includes novel approaches for detecting the above diseases or the individual susceptibility to the above diseases by using plasma, skin fibroblasts and/or PBMCs. These approaches involve the evaluation of total cholesterol, HDL-cholesterol and LDL-cholesterol levels in plasma, and of FC and CE content, and of cholesterol trafficking and metabolism indicators in both non-proliferating and proliferating peripheral cells.

Diagnosis of disease, or of risk of disease, will be made through the comparison of the relative values of the above parameters in suspected cases with those of appropriate standardized age-matched controls.

Therefore it is an object of the present invention, a method to diagnose and/or to make prognostic predictions and/or to monitor the efficacy of a therapy of a proliferative or conformational disease, or to establish the state of ageing in a subject, comprising the steps of:

a) collecting a blood sample from the subject;
b) separating plasma and isolating peripheral blood lymphocytes (PBMCs) from the blood sample;
c) measuring the level of HDL-cholesterol in the plasma;
d) culturing PBMCs and promoting their proliferation by stimulation with an appropriate efficient amount of a stimulating agent to get a sufficient amount of cultured PBMCs;
e) isolating the lipid fraction from the cultured PBMCs;
f) determining the amount of free and esterified cholesterol from the isolated lipid fraction of cultured PBMCs;
g) detecting at least one mRNA and/or its translated protein involved in intracellular cholesterol homeostasis in the cultured PBMCs; and, optionally
h) detecting at least one mRNA and/or its translated protein involved in the pathogenesis of specific proliferative or conformational diseases in the cultured PBMCs; and, optionally
i) detecting at least one mRNA and/or its translated protein of pro-inflammatory cytokines in the cultured PBMCs;
j) comparing the results of steps c), f), g), h), i) with standardized values from an age-matched control sample.

Preferably, the proliferative disease is selected from the group of: atherosclerosis, restenosis after angioplastic, hematologic neoplasms, solid tumors. More preferably, hematologic neoplasms are selected from the group of: Hodgkin and non-Hodgkin lymphomas, acute and chronic leukemias, eritroleukemias, mielomas or policytemias. Even more preferably, the solid tumors are selected from the group of: brain, headneck, nasopharyngeal, breast, ling, gastrointestinal, colon, kidney or liver tumor.

Still preferably, the conformational disease is selected from the group of: prion-related disorders, Alzheimer's disease, Parkinson's disease, Huntington disease, amyotrophic lateral sclerosis or spinocerebellar degenerations. More preferably, the prion-related disorders are selected from the group of: Creutzfeldt-Jacob disease, new variant Creutzfeldt-Jacob disease, Gerstmann-Straussler Sheinker syndrome, fatal familial insomnia, bovine spongiform encephalopathy, scrapie, chronic wasting disease, feline spongiform encephalopathy.

Preferably, the stimulating agent is a mitogenic agent. More preferably, the mitogenic agent is phytohemagglutinin or Concanavalin A.

In one embodiment, steps d)-j) of the method described above are substituted by the following steps:

d′) isolating fibroblasts from the subject;
e′) culturing, synchronizing and optionally stimulating isolated fibroblasts to obtain a sufficient amount of cells;
f′) isolating lipid fraction from cultured fibroblasts;
g′) determining the amount of free and esterified cholesterol in the isolated lipid fraction or in the cultured fibroblasts;
h′) detecting at least one mRNA and/or translated protein involved in intracellular cholesterol homeostasis in the cultured fibroblasts; and, optionally
i′) detecting at least one mRNA and/or translated protein involved in the pathogenesis of specific proliferative or conformational diseases in the cultured fibroblasts; and, optionally
j′) detecting at least one mRNA or translated protein of pro-inflammatory cytokines in the cultured fibroblasts;
k′) comparing the results of steps c), g′), h′), i′) and j′) with standardized values from an age-matched control sample.

Preferably, the synchronizing fibroblasts step of e′) is performed by serum deprivation. More preferably, the stimulating fibroblasts step of e′) is performed by addition of fetal calf serum or at least one mitogen. Preferably, the mitogen is β-FGF.

Still preferably, the esterified cholesterol is measured by staining of cells with oil red O.

Yet preferably, the mRNA and/or translated protein involved in intracellular cholesterol homeostasis is comprised in the group of LDL-R, HMGCoA-R, SREBP2, MDR1, ACAT-1, Caveolin-1, nCEH and ABCA1.

Preferably, the mRNA and/or translated protein involved in the pathogenesis of the proliferative and conformational is comprised in the group of: APP, Neprilysin, β-secretase; PrP protein; tumor suppressor genes such as p16, p53, PTEN and oncogenes such as cMyc, Cyclin D1, ErbB2, EGF-R and Bcl2.

More preferably, the mRNA and/or translated protein of pro-inflammatory cytokines is selected in the group of: Tumour Necrosis Factor alpha (TNFα), Interleukin-1 alpha (IL-1α) and Interferon-gamma (IFNγ).

Preferably, the methods above described further comprises the step of determining the ApoE aplotype.

It is an object of the invention a method to screen drugs for therapeutical effect of a proliferative or conformational disease, comprising the steps of:

a) collecting a blood sample from an affected subject;
b) separating plasma and isolating peripheral blood lymphocytes (PBMCs) from the blood sample;
c) culturing PBMCs and promoting their proliferation by stimulation with an appropriate efficient amount of a stimulating agent to get a sufficient amount of cultured PBMCs;
d) incubating PBMCs with each of drugs at appropriate conditions and dosages;
e) detecting at least one mRNA and/or its translated protein involved in intracellular cholesterol homeostasis in the cultured PBMCs;
f) detecting at least one mRNA and/or its translated protein involved in the pathogenesis of specific proliferative or conformational diseases in the cultured PBMCs;
g) detecting at least one mRNA and/or its translated protein of pro-inflammatory cytokines in the cultured PBMCs;
h) comparing the results of steps e), f), g) with reference drugs and proper controls.

A further object of the invention is a method to screen drugs for therapeutical effect of a proliferative or conformational disease, comprising the steps of:

a) isolating fibroblasts from the subject;
b) culturing, synchronizing and optionally stimulating isolated fibroblasts to obtain a sufficient amount of cells;
c) incubating cultured fibroblasts with each of drugs at appropriate conditions and dosages;
d) detecting at least one mRNA and/or translated protein involved in intracellular cholesterol homeostasis in the cultured fibroblasts;
e) detecting at least one mRNA and/or translated protein involved in the pathogenesis of specific proliferative or conformational diseases in the cultured fibroblasts;
f) detecting at least one mRNA or translated protein of pro-inflammatory cytokines in the cultured fibroblasts;
g) comparing the results of steps d), e), f) with reference drugs and proper controls.

Another object of the invention is a method to assess drugs response profile of a subject affected by a proliferative or conformational disease, comprising the steps of:

a) collecting a blood sample from the affected subject;
b) separating plasma and isolating peripheral blood lymphocytes (PBMCs) from the blood sample;
c) culturing PBMCs and promoting their proliferation by stimulation with an appropriate efficient amount of a stimulating agent to get a sufficient amount of cultured PBMCs;
d) incubating PBMCs with each of drugs at appropriate conditions and dosages;
e) detecting at least one mRNA and/or its translated protein involved in intracellular cholesterol homeostasis in the cultured PBMCs;
f) detecting at least one mRNA and/or its translated protein involved in the pathogenesis of specific proliferative or conformational diseases in the cultured PBMCs;
g) detecting at least one mRNA and/or its translated protein of pro-inflammatory cytokines in the cultured PBMCs;
h) comparing the results of steps e), f), g) with reference drugs, proper controls and among tested drugs.

A further object of the invention is a method to assess drugs response profile of a subject affected by a proliferative or conformational disease, comprising the steps of:

a) isolating fibroblasts from the subject;
b) culturing, synchronizing and optionally stimulating isolated fibroblasts to obtain a sufficient amount of cells;
c) incubating cultured fibroblasts with each of drugs at appropriate conditions and dosages;
d) detecting at least one mRNA and/or translated protein involved in intracellular cholesterol homeostasis in the cultured fibroblasts;
e) detecting at least one mRNA and/or translated protein involved in the pathogenesis of specific proliferative or conformational diseases in the cultured fibroblasts;
f) detecting at least one mRNA or translated protein of pro-inflammatory cytokines in the cultured fibroblasts;
g) comparing the results of steps d), e), f) with reference drugs, proper controls, and among tested drugs.

Another object of the invention is a kit for measuring the esterified cholesterol in the cell including means for cell staining with oil red O.

A further object of the invention is a kit for the detection of at least one mRNA involved in intracellular cholesterol homeostasis including:
means for reverse transcription;
means for specific amplification of cDNA or fragments thereof comprised in the group of: LDL-R, HMGCoA-R, SREBP2, MDR1, ACAT-1, Caveolin-1, nCEH and ABCA1;
detection means.

Another object of the invention is a kit for the detection of at least one mRNA involved in the pathogenesis of the proliferative or conformational diseases including:
means for reverse transcription;
means for the specific amplification of cDNA or fragments thereof comprised in the group of: APP, Neprilysin, β-Secretase, PrP protein, tumor suppressor genes such as p16, p53, PTEN and oncogenes such as cMyc, Cyclin D1, ErbB2, EGF-R and Bcl2;
detection means.

Another object of the invention is a kit for the detection of at least one mRNA of pro-inflammatory cytokines, including:
means for reverse transcription;
means for the specific amplification of cDNA or fragments thereof comprised in the group of cytokines: Tumour Necrosis Factor alpha (TNFα), Interleukin-1 alpha (IL-1α) and Interferon-gamma (IFNγ);
detection means.

Another object of the invention is a kit for the detection of at least one protein involved in intracellular cholesterol homeostasis including at least a ligand specific for one of the proteins comprised in the group: LDL-R, HMGCoA-R, SREBP2, MDR1, ACAT-1, Caveolin-1, nCEH and ABCA1.

Still object of the invention is kit for the detection of at least one protein involved in the pathogenesis of the proliferative or conformational diseases including at least a ligand specific for one of the proteins comprised in the group of: APP, Neprilysin and β-Secretase for Alzheimer's disease; PrP protein for prion-related diseases; tumor suppressor genes such as p16, p53, PTEN and oncogenes such as cMyc, Cyclin D1, ErbB2, EGF-R and Bcl2 for hematologic neoplasms and solid tumors.

A further object of the invention is a kit for the detection of at least one pro-inflamatory cytokine, including at least one ligand for cytokines comprised but not limited to the group of: Tumour Necrosis Factor alpha (TNFα), Interleukin-1 alpha (IL-1α) and Interferon-gamma (IFNγ).

Another object of the invention is a diagnostic platform to diagnose, and/or to make prognostic predictions, and/or to monitor the efficacy of a therapy, and/or to screen drugs for therapeutical effect, and/or to assess drugs response profile of a subject affected by a proliferative or conformational disease, or to establish the state of ageing in a subject, including all of kits according to claims 22 to 28.

In the present invention proliferative and conformational diseases comprise, but are not limited to, the diseases indicated in Table 2.

TABLE 2
Proliferative and conformational diseases
Proliferative diseases
Atherosclerosis
Restenosis after angioplastic
Hematologic neoplasms
Hodgkin and non-Hodgkin lymphomas
Acute and cronic leukemias
Eritroleukemias
Mielomas
Policytemias
Solid tumors
Brain
Headneck
Nasopharingeal
Breast
Lung
Gastrointestinal
Colon
Kidney
Liver
Conformational diseases:
Prion related disorders (TSE)
Creutzfeldt-Jacob Disease (CJD)
New Variant Creutzfeldt-Jacob Disease (vCJD)
Gerstmann-Straussler Sheinker Syndome (GSS)
Fatal Familial Insomnia (FFI)
Bovine Spongiform Encephalopathy (BSE)
Scrapie
Chronic Wasting Disease (CWD)
Feline Spongiform Encephalopathy (FSE)
Alzheimer's disease
Parkinson's disease
Huntington's disease
Amyothropic Lateral Sclerosis
Ataxia Spinocerebellar

The invention will be now described by non limiting examples referring to the following figures:

FIG. 1: Intracellular cholesterol homeostasis.

Intracellular cholesterol derives from i) endogenous neosynthesis in the ergastoplasmic reticulum (ER) through the activity of HMGCoA-reductase (HMGCoA-R) (1); ii) circulating low density lipoproteins (LDL) (2), which are first internalised via LDL receptors (a) and then hydrolytically processed in lysosomes to generate free cholesterol (FC) through the activity of acid cholesterol ester hydrolase (aCEH) (b). Most of the newly synthesized FC, or LDL-bound FC released in the lysosomes, rapidly emerges at cell surface caveolae, from where it may be used for cellular functions (c). If plasma membrane FC exceeds a threshold level, the excess FC is rapidly transported to the ER (d) by a P-glycoprotein (MDR1-Pgp) encoded by the multidrug resistance (MDR1) gene. Then, the ER-resident enzyme acyl-coenzyme A-cholesterol-acyl-transferase (ACAT) (e) converts FC into cholesteryl esters (CE), which are stored in cytoplasmic droplets. If cells require cholesterol, CE is re-hydrolyzed to FC by the enzyme neutral cholesterol-esters-hydrolase (nCEH) (f) and transported to the plasma membrane by caveolin-1. If in excess, FC is eliminated from the cells through an efflux pathway spanning from the ER to the plasma membrane and involving caveolin-1, the ABCA1 receptor, and plasma HDLs (g).

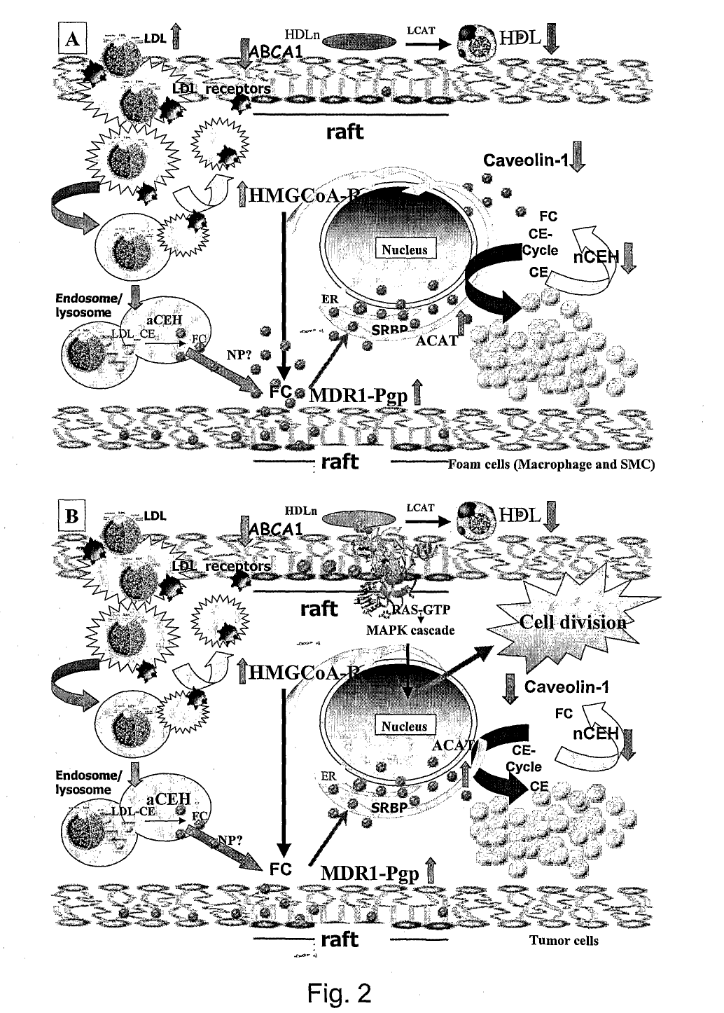

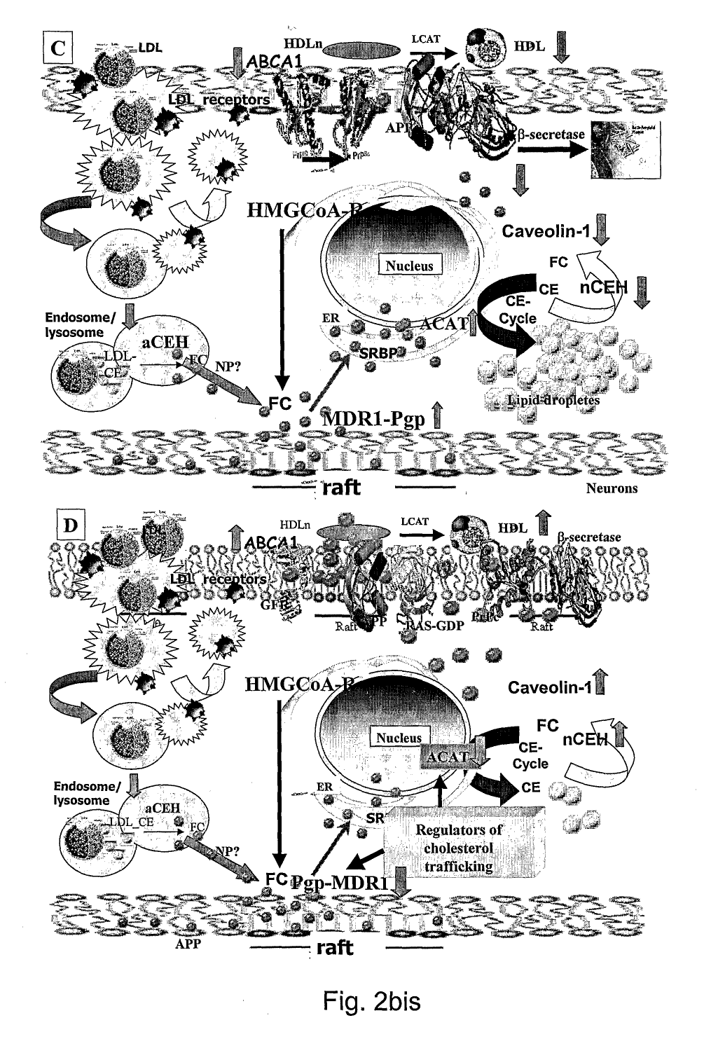

FIGS. 2 and 2bis: Alterations of cholesterol homeostasis in pathologic conditions.

When alterations in cholesterol homeostasis occur, such as excessive cholesterol synthesis and uptake, more cholesterol is transported to the ER by MDR1-Pgp. Cholesterol overloading in the ER leads to activation of the ACAT enzyme which, in turn, esterifies cholesterol. Cholesterol esters (CE) are then accumulated in the cytoplasm as lipid droplets, and foam-like cells are formed (Schemes 2 A, B, and C). On the other hand, excessive accumulation of cholesterol in the form of esters reduces the cholesterol pool that can be transported by caveolin-1 to the plasma membrane (rafts) for excretion. This leads to a decreased cholesterol efflux via ABCA-1 receptors and, thus, to lower HDL-cholesterol levels in plasma (Schemes 2 A, B, and C). Lower levels of FC into the rafts may also lead to a continuous activation of raft-protein functions in membrane signalling, protein and lipid processing and transport, thus triggering a variety of pathologic processes including atherosclerosis (FIG. 2, Scheme A), tumors (FIG. 2, Scheme B) and AD and prion-related (TSE) diseases (FIG. 2bis, Scheme C). Treatment with modulators of cholesterol metabolism/trafficking could restore the normal raft-protein functions by re-establishing the intracellular cholesterol homeostasis (FIG. 2bis, Scheme D).

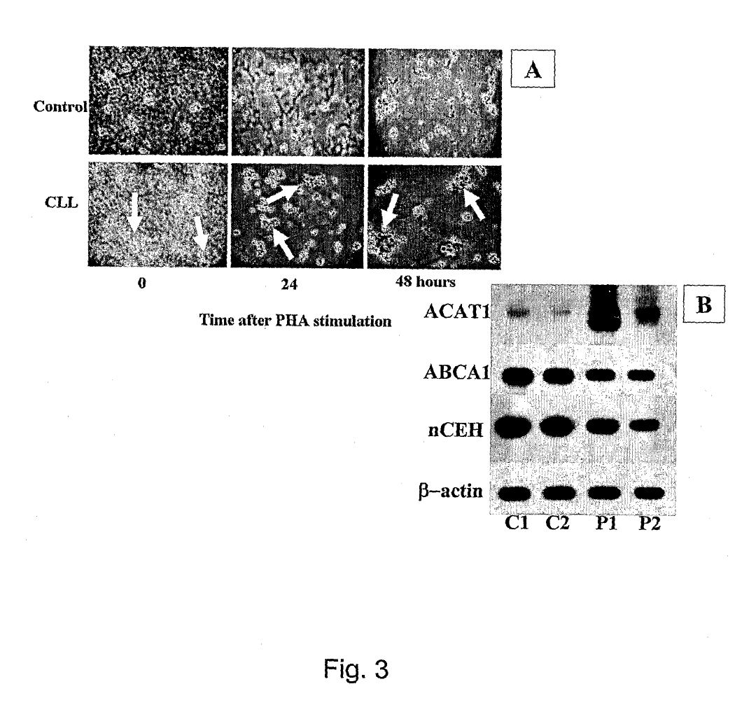

FIG. 3. Neutral lipid content and mRNA expression levels of genes involved in cholesterol metabolism and trafficking in PBMCs from patients with Chronic Lymphocytic Leukaemia (CLL). A) PBMCs from CLL patient and from an healthy individual (control) stained for neutral lipid content by the ORO method (indicated by the arrows, see section Materials and Methods) at the indicated times after PHA-stimulation. B) mRNA levels of the indicated genes in PBMCs from CLL patients (P1 and P2) and from healthy individuals (C1 and C2) by RT-PCR (see Materials and Methods). Beta-actin was used as an internal standard.

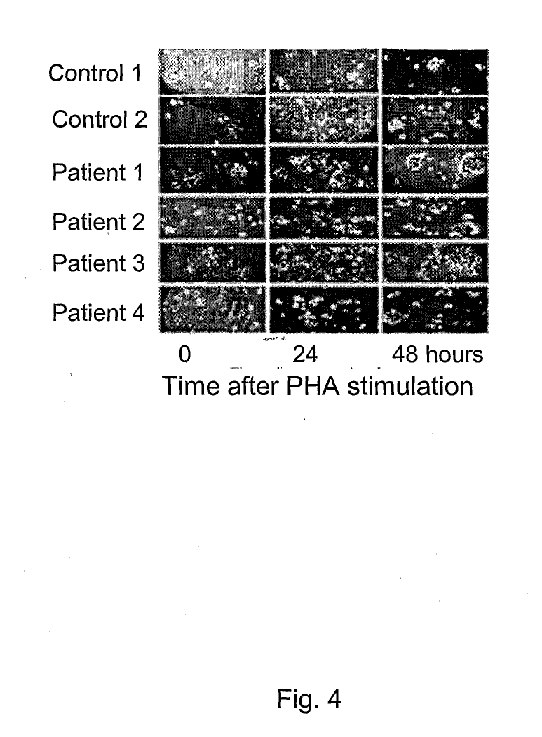

FIG. 4. Neutral lipid content in PBMCs from patients with atherosclerotic plaques. PBMCs from atherosclerotic patients and from healthy individuals (controls 1 and 2) stained for neutral lipid content by the ORO method at the indicated times after PHA-stimulation.

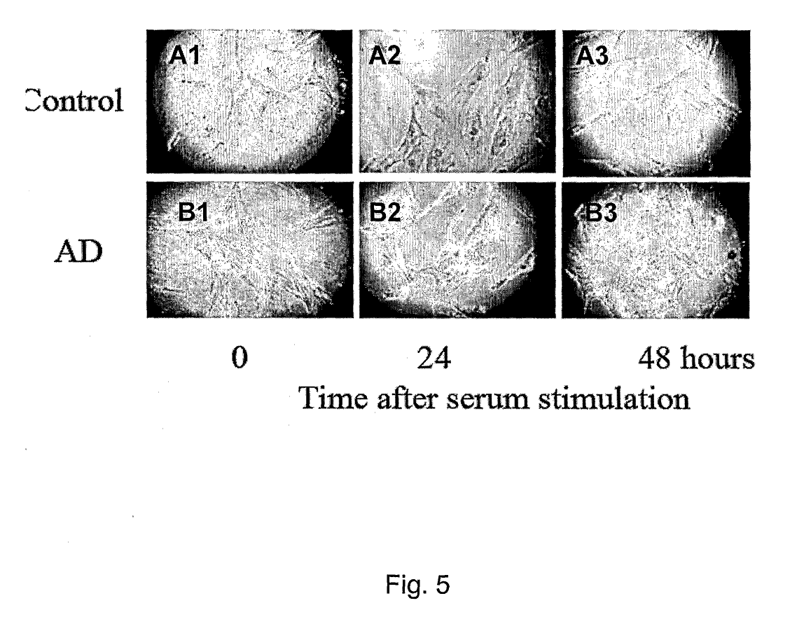

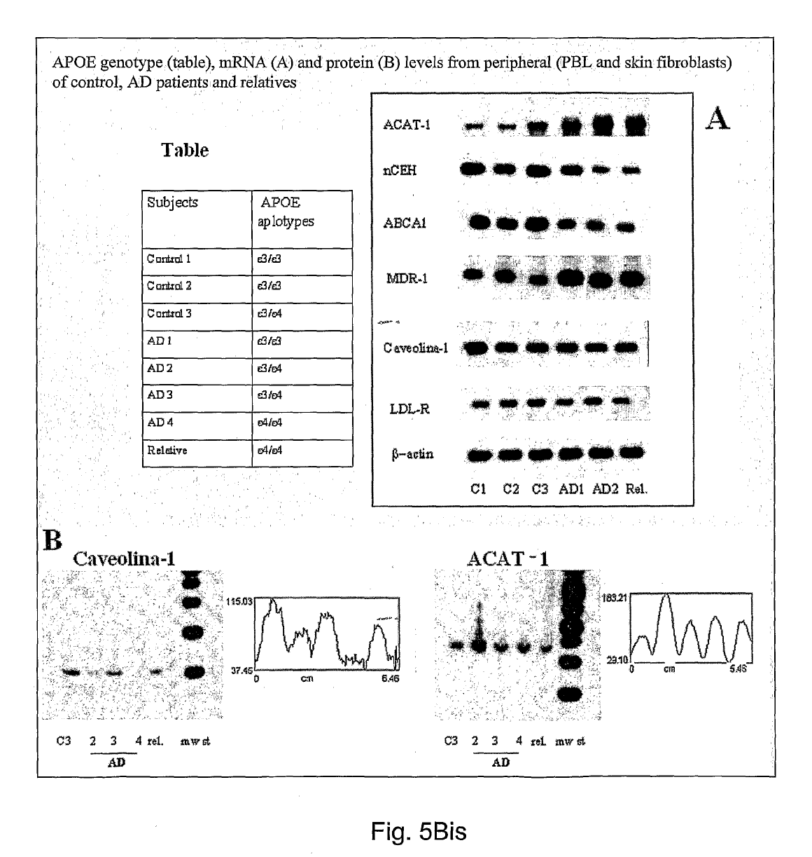

FIG. 5. Neutral lipid content in skin fibroblasts from AD patients (AD). Skin fibroblasts from an AD patient and from a healthy control individual were stained for neutral lipid content by the ORO method at 0 (A1, B1), 24 (A2, B2) and 48 (A3, B3) hours after serum stimulation.

FIG. 5 Bis. Protein and mRNA levels of genes involved in cholesterol metabolism and trafficking in skin fibroblasts from AD patients (AD).

Panel A shows ApoE genotype (table) and mRNA levels of ACAT-1, nCEH, ABCA-1, MDR1, Caveolin-1, LDL-R and β-actin genes in skin fibroblasts from AD patients (AD), their relatives (Rel) and control healthy donors (C). Panel B shows Western blotting analysis of caveolin-1 and ACAT-1 expression in skin fibroblasts from AD, their relatives and controls.

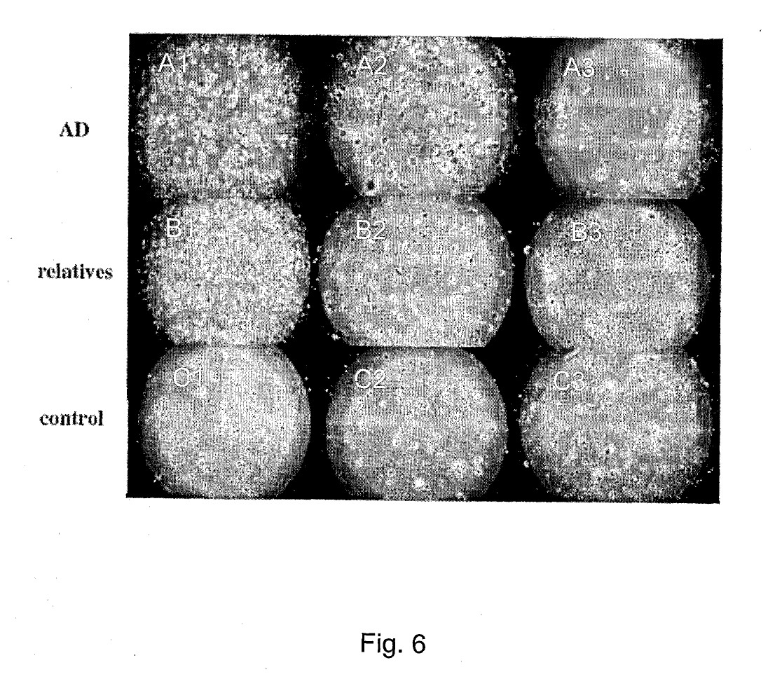

FIG. 6. Lipid droplets in PBMC from AD patients, their relatives and controls.

PBMC from AD patients (A1-A3), their relatives (B1-B3) and healthy individuals (C1-C3, control group) stained for neutral lipid by the ORO method at 0 (A1, B1, C1), 24 (A2, B2, C2) and 48 (A3, B3, C3) hours after PHA stimulation.

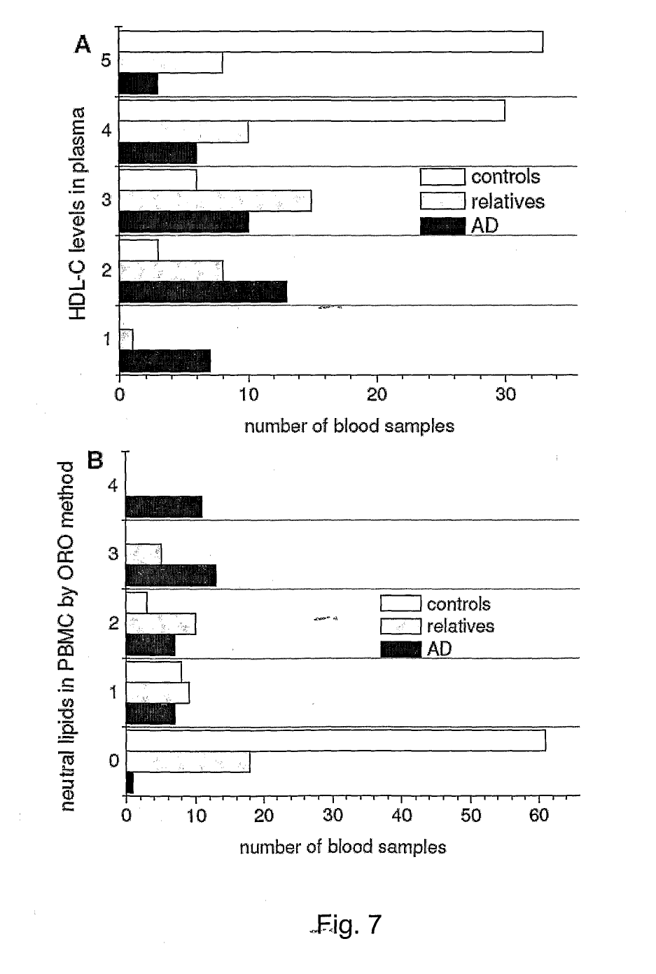

FIG. 7: HDL-cholesterol levels in plasma and neutral lipid content in PBMC from AD patients, their relatives and controls. A) HDL-C levels in plasma were stratified into 5 classes (0-20, 21-30, 31-40, 41-50, >50 mg/dl) of increasing values, expressed in mg/dl as determined by the enzymatic method (see Materials and Methods). B) Intracellular neutral lipids in PBMC, stained with ORO, were quantified based on the intensity of the lipid bound red color by two different methods. The degree was further graded into 5 classes 1 (0), 2(+), 3(++), 3 (+++) and 4 (++++) based on intensity measurements.

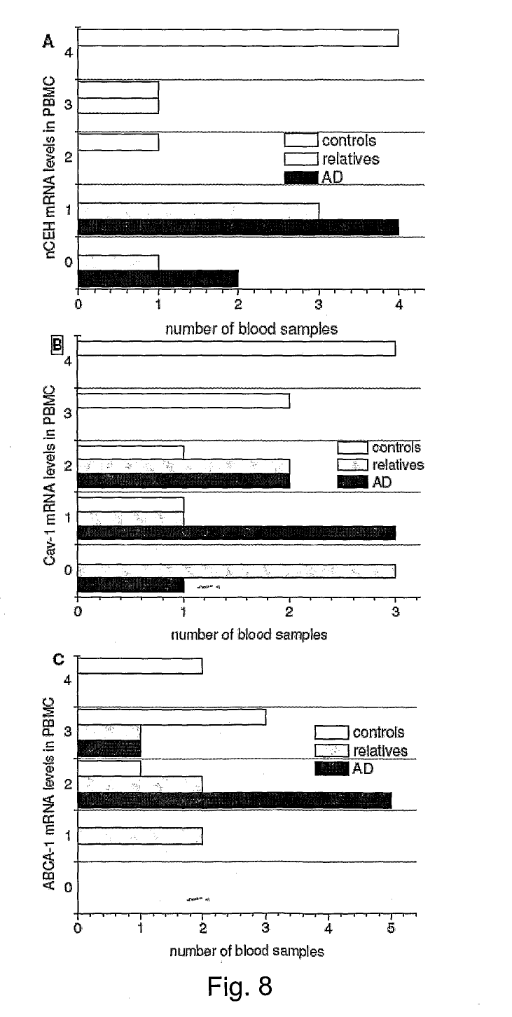

FIG. 8: Expression levels of mRNA of genes involved in cholesterol metabolism and trafficking in PBMC from patients with Alzheimer's disease, their relatives and controls. A) nCEH, B) Cav-1, C) ABCA1. Quantification of autoradiograms was obtained by densitometric analysis through the Scion Image software (NIH). Values were normalized against expression levels of the housekeeping gene β-actin and stratified into 5 classes (0 to 5) of increasing values (see Materials and Methods).

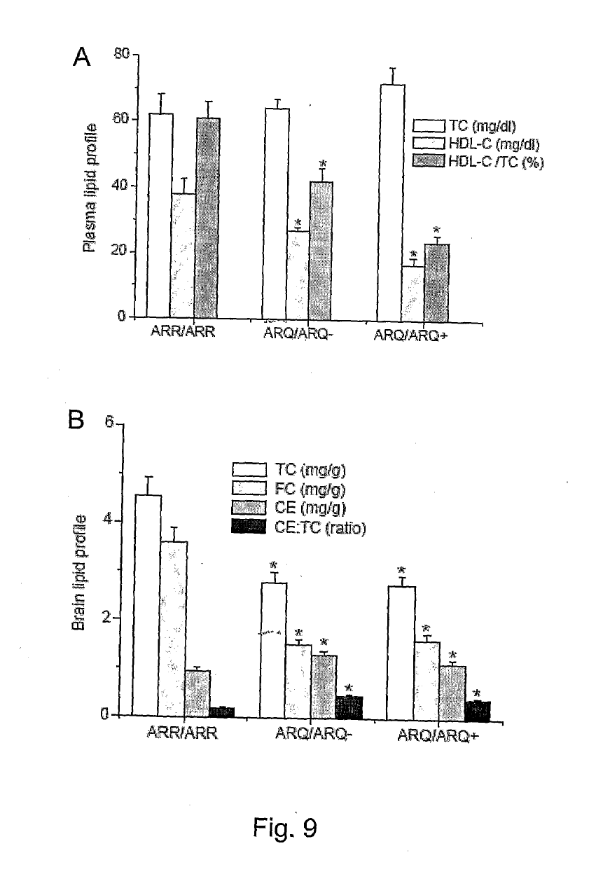

FIG. 9: A) Lipid profiles in plasma samples and B) cholesterol content in brain tissue from sheep with scrapie-susceptible (ARQ/ARQ) genotype, either infected naturally or experimentally with scrapie (ARQ/ARQ+) or not infected (ARQ/ARQ−) and sheep with scrapie-resistant (ARR/ARR) genotype. Student's t-test, * P<0.05 vs ARR/ARR.

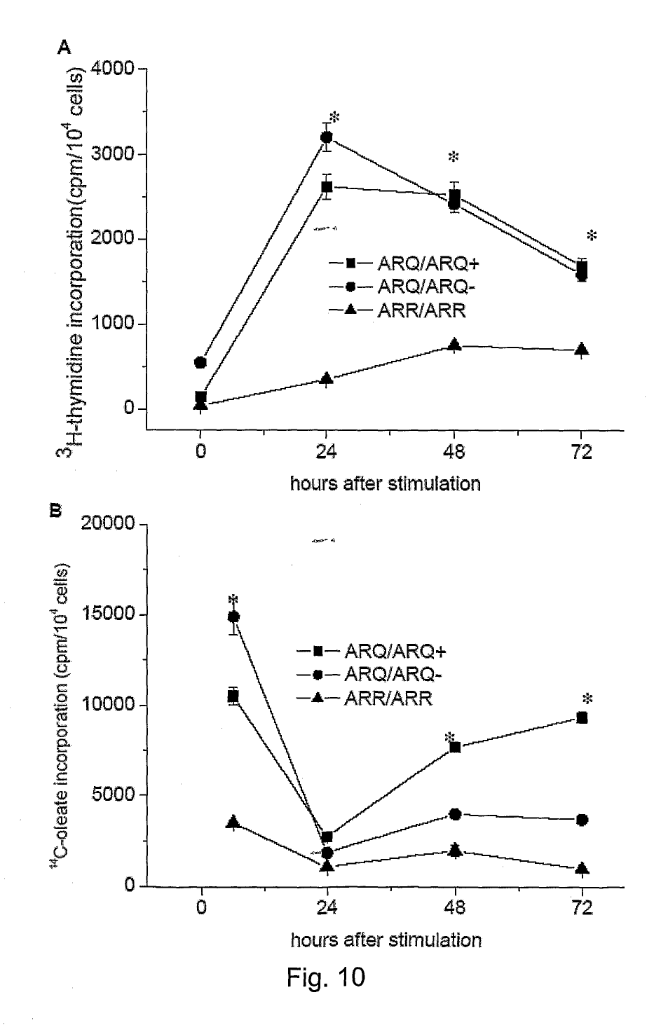

FIG. 10: Cell growth and cholesterol esterification in FCS-stimulated skin fibroblasts from sheep with scrapie-susceptible (ARQ/ARQ) genotype, either infected with scrapie (ARQ/ARQ+) or not infected (ARQ/ARQ−) and sheep with scrapie-resistant (ARR/ARR) genotype. A) Thymidine incorporation into DNA. B) ¹⁴C-Oleate incorporation into CE. Student's t-test, * P<0.05 vs ARR/ARR.

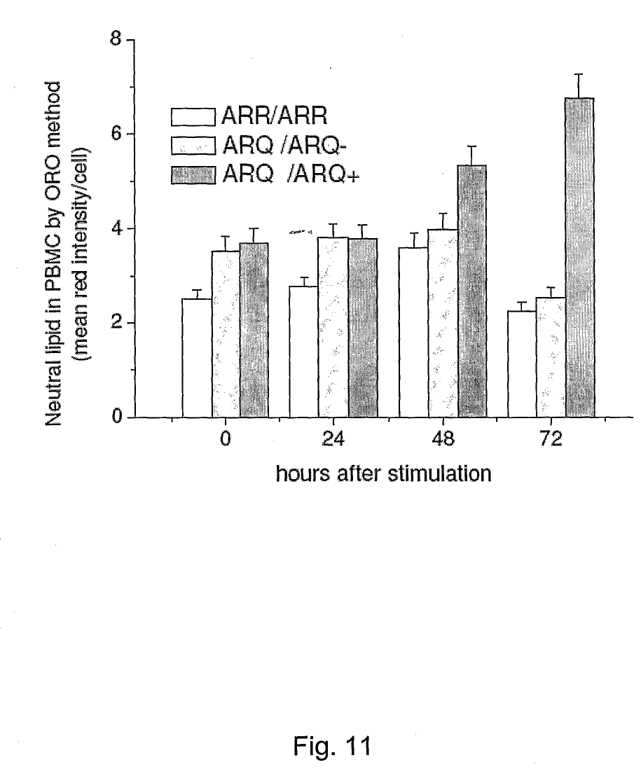

FIG. 11: Neutral lipid content in FCS-stimulated skin fibroblasts from sheep with scrapie-susceptible (ARQ/ARQ) genotype, either infected with scrapie (ARQ/ARQ+) or not infected (ARQ/ARQ−) and sheep with scrapie-resistant (ARR/ARR) genotype. Intracellular neutral lipids in skin fibroblasts were stained with ORO, and quantified by a method based on the intensity of the lipid bound red color.

### MATERIALS AND METHODS

### Patients and Cells Sources

Individuals with clinical diagnosis or at risk of AD (or TSE). Clinical diagnoses of AD are made according to the NINCDS-ADRDA criteria. Additional measures include the MMSE and Dementia Severity Rating Scale (DSRS). Routine laboratory studies, including magnetic resonance imaging, are performed to rule out other causes of cognitive impairment. The Reisberg Global Deterioration Scale (GDS) is used to indicate the severity of the cognitive impairment of AD patients. Abnormal GDS levels start from level 3 and maximal deterioration grade corresponds to level 7. First degree relatives of AD patients of different ages and with no cognitive decline are recruited in the study after informed consent. An age-matched group of control subjects with no cognitive decline is recruited at affiliated hospitals or from blood donor lists (AVIS).

Peripheral blood Mononuclear cells (PBMCs) are collected from peripheral blood samples Dermal biopsies are obtained from the upper forearm of the subjects by a 2-mm punch after local anesthesia with 2% xylocaine.

Eighteen patients with chronic lymphocytic leukemia (CLL) (aged 45-65 years) and twelve patients with acute lymphocytic leukemia (ALL) (aged 40-60 years) were recruited at diagnosis in local hospitals. Fifteen healthy age-matched subjects were also recruited as controls. Ten patients (7 with CLL and 3 with ALL) were randomly chosen to perform kinetic and molecular analyses. Informed written consent was obtained from all patients and healthy controls before initiating the study according to the policies of the hospitals Institutional Review Boards.

All subjects enrolled in the various studies did not receive any pharmacological treatment prior to blood or punch sampling.

### Sheep

10 naturally scrapie-affected Sarda breed sheep with the scrapie-susceptible ARQ/ARQ genotypes and 10 experimentally scrapie-infected sheep with ARQ/ARQ genotype were used (all named as ARQ/ARQ+). All sheep were at the terminal clinical stage of the disease. The sheep were euthanized with a barbiturate followed by 4 ml of embutramide and mebenzonico-iodide (Hoechst Roussel Vet). Peripheral blood samples and skin biopsies were collected from the animals. The brains were collected and transverse sections taken for PrPSc detection by Western Blot. The same procedure was performed on 10 scrapie-resistant ARR/ARR genotype sheep and 10 ARQ/ARQ scrapie-free sheeps (ARQ/ARQ−).

### Isolation of PBMCs and Fibroblasts

PBMCs are collected from peripheral blood of patients and controls and separated by Ficoll-Hypaque density gradient. After extensive washings, cells are resuspended (1×10⁶ cells/ml) in RPMI-1640 with 10% FCS and incubated overnight. For assay purposes, 2×10⁵ cells/ml nonadherent cells (lymphocytes) are incubated at 37° C. in RPMI-1640 10% FCS supplemented with PHA (Phythohemoagglutinin, 10 μg/ml, cat. number L8902, SIGMA). Viability is evaluated after a time course by counting cells using trypan blue exclusion. Cells are harvested at different time points of incubations.

For fibroblasts isolation, biopsies are plated into 6 well plates for 2 hours. After 2 hours of adhesion, a few drops of Dulbecco's modified Eagle's medium (DMEM) (Gibco Lab NY, USA) supplemented with 10% fetal bovine serum (FBS) (Sigma), 100 U/ml penicillin/streptomycin (Sigma), and fungizone (Life Technologies, Inc.) covering each fragment are added. The next day, the tissue fragments are covered with culture medium and maintained in a humidified incubator (37° C., 5% CO₂). The medium is changed every 2 days. After 5 to 6 days, fibroblasts begin to proliferate from the fragment margin (“halo of cells”) and create a monolayer of spindle-shaped cells. After 4 weeks, fibroblasts are purified by repeat trypsinization (trypsin-EDTA-0.05%/0.02%) and passaging to achieve a homogenous population of spindle cells. Purified fibroblasts are washed two times with PBS and centrifuged. 1×10⁶ cells are then seeded into 25 cm² culture flask and grown to confluence. At this time cells are used for “in vitro” staining experiments, or transferred into vials containing cryo-preservation medium at a density of 1×10⁷ cells/ml. After swift freezing, vials are transferred into liquid nitrogen for long-term storage. When needed for analysis cryopreserved cells are removed from liquid nitrogen and cultured as described above.

For analysis, skin fibroblasts are plated at a density of 5000 cell/cm² in 6 well plates and then incubated for 48 h in MEM 199 containing 0.2% FCS to force cells into a quiescent state. Before each assay, quiescent cells are stimulated to proliferate synchronously by adding FCS (10%) or in alternative a potent mytogen, such as β-FGF (β-fibroblast growth factor), and incubated for 12, 24, 48 and 72 hours in presence or in absence of different drugs.

All assays are conducted using fibroblasts between passages two to four.

### ³H-Thymidine Incorporation

Cell proliferation was measured by ³H-thymidine incorporation. Cells were labeled with 3H-thymidine (2.5 μCi/ml) during the last 6 hours of culture and harvested at the time points indicated, see FIG. 10. Cells were rinsed twice with ice-cold PBS, washed with 5% cold TCA and lysed with 1M NaOH. The amount of radioactivity was measured by a Beckman β counter (Palo Alto, Calif.) using Ultima Gold as the scintillation fluid. An aliquot of cell lysate was processed for protein content. Any toxic drug effect was excluded by trypan blue uptake.

### Cholesterol Esterification

Cholesterol esterification was evaluated by incubating cells for 6 hours in medium containing [1-¹⁴C] oleic acid (Dupont, NEN 55 mCi/mmol), bound to bovine serum albumin (BSA). After incubation cells were washed with PBS and lipids extracted with acetone. Lipid subclasses were separated by thin layer chromatography (TLC) on kiesegel plates using a solvent system containing n-heptane/isopropyl ether/formic acid (60:40:2, v/v/v). Cholesterol ester bands were identified by comparison with reference standard run simultaneously side-by-side and visualized under iodine vapors. For scintillation counting, the bands were excised and added directly to counting vials containing 10 ml Econofluor (DuPont NEN) liquid scintillation fluid.

### Molecular Biology Assays

The molecular characterization of the aplotype for APOE, is performed on DNA extracted from lymphocytes obtained from peripheral blood samples. DNA is purified according to the QiAmp Blood kit procedure (Quiagen), followed by PCR amplification using sets of primers and standard experimental conditions.

Determination of relative mRNA expression levels for: SREBP2, LDL-R, HMG-CoA-R, MDR1-Pgp, ACAT-1, nCEH, caveolin-1 ABCA-1, APP, Neprilysin, β-secretase, PrP, p16, p53, PTEN, cMyc, Cyclin D1, ErbB2, EGF-R, Bcl2, TNFα, IL-1α and IFNγ is performed on samples of total RNA, purified with the TRIZOL reagent (GIBCO) according to established procedures, by a macroarray system.

### Preparation of Macroarrays

Macroarrays are prepared by printing purified PCR products, suspended in DR.DiY spotting buffer, using the Fast Spotter macroarrayer (Dr, Chip Biotechnologies). Printing is done on aminosilane-treated slides (ex. CMT-GAPS™ from Corning). After printing, slides are allowed to dry at room temperature and then UV-crosslinked with a UVC 500 crosslinker (Hoefer) and used immediately or stored desiccated at room temperature.

Preparation of Labelled cDNAs (in Each of 2 Separate 0.5 ml Eppendorf Tubes)

| | | |
|---|---|---|
| PolyA RNA (from either the sample or control) | 2 | μg |
| oligo - (dT) primer (18-20mer) 1 μg/μl | 2 | μl |
| DEPC-treated water to 10 μl | X | μl |
| Heat at 70° C. for 10 min and chill on ice | | |

### Preparation of First Strand Reaction Mix

| | | |
|---|---|---|
| 5X first strand buffer | 6 | μl |
| dNTP mix (10 mM each) | 1.5 | μl |
| DTT (0.1M) | 3 | μl |
| RNase out | 1 | μl |
| DEPC-treated water to 30 μl | 18.5 | μl |
| To each tube containing the denatured mRNA are added | | |
| First strand reaction mix | 15 | μl |
| Cy3 or Cy5 dUTP 1 mM | 3 | μl |
| Superscript ™ II reverse transcriptase (200 U/μl) | 2 | μl |

The reactions are mixed, the tubes are wrapped in aluminum foil and incubated at 42° C. for 2 hours.

Tubes are then pulse-spun and 1.5 μl of EDTA are added to stop the reaction

### Probe Cleaning

Labeling reactions are loaded onto filtration columns (ex. Microspin™ G-50, Amersham Pharmacia) and washed according to the protocol provided by the manufacturer. Eluted probes are collected in clean tubes, wrapped in foil, and stored at −20° C. if not used immediately.

### Array Hybridization:

Equal volumes (10 μl) of the two probes are combined in a 0.5 ml Eppendorf tube and then are added:

| | |
|---|---|
| COT1 DNA (20 μg/μl) | 1 μl |
| Poly A RNA (20 μg/μl) | 1 μl |

Probes are denatured by heating at 95° C. for 3 min. and then combined with an equal volume of hybridization buffer (10×SSC, 50% formamide, 0.2% SDS).

Just before use, slides are prehybridized in a Coplin jar with Pre-hyb buffer (5×SSC, 0.1% SDS and 1% BSA) for 30 min at 42° C.

Slides are dipped in filtered Milli-Q water and then in isopropanol and allowed to dry at room temperature. Thirty μl of probe mix are overlaid onto the macroarray and covered with a 22×60 mm hydrofobic coverslip. Slides are set in Hybridization chambers which are then sealed with the lid and placed in a water bath at 42° C. and incubated for 16-20 hours in the dark. After hybridization slides are placed in a Petri dish, submerged in wash buffer (1×SSC, 0.2% SDS) at 42%. The coverslips are lifted gently and removed while the slides are submerged and these are then washed with stringency buffer (0.1×SSC, 0.2% SDS). After washing the slides are allowed to dry at room temperature and immediately scanned with a GenePix® 4000B Fluorescent scanner and analyzed with the GenePix Pro software. Relative levels of expression are calculated as red-to-green signal ratio, after normalization and background subtraction.

TABLE 3

Primers and conditions used in the invention for mRNA quantification of genes
involved in intracellular cholesterol metabolism and trafficking, by macroarrays.

| | Accession | | | |
|---|---|---|---|---|
| Gene | No. | Forward | Reverse | Conditions |
| ACAT1 | NM_003101 | 5′AGCAGAGGCAGAGGA | 5′GCACACCTGGCAAGA | 466 bp 95° C. (30″) 58° C. (50″), |
| Used for | | ATTGA3′ | TGGAG 3′ | and 72° C. (60″), (40 cycles) |
| atherosclerosis | | Seq ID 1 | Seq ID 2 | |
| diagnosis | | | | |
| MDR1 | M14758 | 5′CCCATCATTGCAATAG | 5′GTTCAAACTTCTGCTC | 167 bp 94° C. (30″) 55° C. (60″), |
| Used as | | CAGG3′ | CTGA3′ | and 72° C. (50″), (30 cycles) |
| determinant | | Seq ID 3 | Seq ID 4 | |
| of | | | | |
| responsiveness | | | | |
| to therapy and | | | | |
| survival in | | | | |
| some cancers. | | | | |
| nCEH | M85201 | 5′CTTGTAAACTTGAGTT | 5′GTAGGAAGTAACCAC | 151 bp 94° C. (30″) 55° C. (60″), |
| No other | | GGAG3′ | ATTCA3′ | and 72° C. (60″), (30 cycles) |
| known use | | Seq ID 5 | Seq ID 6 | |
| Caveolin-1 | NM_001753 | 5′GAGCGAGAAGCAAGT | 5′ACAGACGGTGTGGAC | 360 bp 94° C. (30″) 55° C. (45″), |
| Used as | | GTACGA3′ | GTAGAT3′ | and 72° C. (120″), (30 cycles) |
| tumoral | | Seq ID 7 | Seq ID 8 | |
| marker | | | | |
| ABCA1 | NM_005502 | 5′TCCTCTCCCAGAGCAA | 5′CTCCACAACACTTCA | 286 bp 95° C. (30″) 62° C. (60″), |
| Used for | | AAAGC3′ | CATGGT3′ | and 72° C. (30″), (30 cycles) |
| Tangier | | Seq ID 9 | Seq ID 10 | |
| disease | | | | |
| diagnosis (a | | | | |
| reverse | | | | |
| cholesterol | | | | |
| transport | | | | |
| deficiency) | | | | |
| SREBP2 | | 5′ATACCAGAATGCAGCT | 5′ATCTGTCTTGATGATC | 244 bp 95° C. (30″) 62° C. (60″), |
| Used for | | ACTA3′ | TGAG3′ | and 72° C. (30″), (30 cycles) |
| cholesterol | | Seq ID 11 | Seq ID 12 | |
| homeostasis | | | | |
| LDL-R | | 5′CAATGTCTCACCAAGC | 5′TCTGTCTCGAGGGGT | 258 bp 95° C. (30″) 62° C. (60″), |
| Used for | | TCTG3′ | AGCTG3′ | and 72° C. (30″), (30 cycles) |
| cholsterol | | Seq ID 13 | Seq ID 14 | |
| homeostasis | | | | |
| HMG-CoA-R | | 5′TACCATGTCAGGGGTA | 5′CAAGCCTAGAGACAT | 246 bp 95° C. (30″) 62° C. (60″), |
| Used for | | CGTC3′ | AATCATC3′ | and 72° C. (30″), (30 cycles) |
| cholesterol | | Seq ID 15 | Seq ID 16 | |
| homeostasis | | | | |
| APP | | 5′TCAGATCCGCTCCCAG | 5′AGAGTCAGCCCCAAA | 313 bp 95° C. (30″) 62° C. (60″), |
| Used for AD | | GTTATG3′ | AGAATGC3′ | and 72° C. (30″), (30 cycles) |
| | | Seq ID 17 | Seq ID 18 | |
| Neprilysin | | 5′CTACCGATTCTTTGGA | 5′CTTCTCTTGCTGAGAA | 404 bp 95° C. (30″) 62° C. (60″), |
| Used for AD | | GTTG3′ | CCAC3′ | and 72° C. (30″), (30 cycles) |
| | | Seq ID 19 | Seq ID 20 | |
| β-secretase | | 5′TCTAAAACTAGGACTG | 5′CTTTAGAGACAGGGA | 202 bp 95° C. (25″) 58° C. (30″) |
| used for AD | | GTGA 3′ | CTGTA3′ | and 73° C. (50″), (30 cycles) |
| disease | | Seq ID 21 | Seq ID 22 | |
| PrP | | 5′ATTGTCACCTAGCAGA | 5′TTGTTCAGTAGCTCA | 341 bp 95° C. (30″) 62° C. (60″), |
| Used for prion | | TAGA3′ | AGTCT3′ | and 72° C. (30″), (30 cycles) |
| disease | | Seq ID 23 | Seq ID 24 | |
| P16 | | 5′ATATGCCTTCCCCCACT | 5′CCCCTGAGCTTCCCTA | 231 bp 95° C. (30″) 50° C. (60″), |
| Tunor marker | | ACC3′ | GTTC3′ | and 72° C. (30″), (30 cycles) |
| | | Seq ID 25 | Seq ID 26 | |
| P53 | | 5′CAGTCTACCTCCCGCCA | 5′CCACAACAAAACACC | 246 bp 95° C. (30″) 47° C. (60″), |
| Tumor | | TAA3′ | AGTGC3′ | and 72° C. (30″), (30 cycles) |
| marker | | Seq ID 27 | Seq ID 28 | |
| PTEN major | | 5′-CTA CTC GAG GCT CCC | 5′-ACG CTC GAG ATA | 460 bp 95° C. (30″) 47° C. (60″), |
| negative | | AGA CAT GAC-3′ | AAA AAA AATTCAG-3′ | and 72° C. (30″), (30 cycles) |
| regulator of the | | Seq ID 29 | Seq ID 30 | |
| PI3K/Akt | | | | |
| signal pathway | | | | |
| cMyc | | 5′ATG CCC CTC AAC GTT | 5′GTG GGC AGC TCG | 564 bp 95° C. (40″) 55° C. (60″), |
| | | AGC TT3′ | AAT TT3′ | and 72° C. (35″), |
| | | Seq ID 31 | Seq ID 32 | |
| Cyclin D1 | | 5′CTGGAGCCCCTGAAAA | 5′CTGGAGAGGAAGCGT | 415 bp 95° C. (30″) 58° C. (60″), |
| Used for the | | AGAGC3′ | GTGAGG3′ | and 72° C. (30″), (30 cycles) |
| control of cell | | Seq ID 33 | Seq ID 34 | |
| cycle | | | | |
| ErbB2 | | 5′- | 5′- | 205 bp 94° C. (30″) 62° C. (60″), |
| Used for the | | CAACCAAGTGAGGCAGG | AGAGGCTGCGGATTGT | and 72° C. (30″), (30 cycles) |
| control of cell | | TCC-3′ | GCGA-3′ | |
| cycle | | Seq ID 35 | Seq ID 36 | |
| EGF-R | | 5′- | 5′- | 205 bp 94° C. (120″) 55° C. (60″), |
| Used for the | | GGAAAAGAAAGGAAACT | AGTTCCCGTGGGTCTAG | and 72° C. (30″), (40 cycles) |
| control of cell | | ACGTGG3′ | AGG-3′ | |
| cycle | | Seq ID 37 | Seq ID 38 | |
| Bcl2 | | 5′GTGAACTGGGGGAGGA | 5′ACAGTTCCACAAAGG | 181 bp 95° C. (30″) 62° C. (60″), |
| Tumor | | TTGT3′ | CATCC3′ | and 72° C. (30″), (30 cycles) |
| marker | | Seq ID 39 | Seq ID 40 | |
| TNFα | | 5′CGGGACGTGGAGCTGG | 5′CACCAGCTGGTTATC | 432 bp 95° C. (30″) 62° C. (60″), |
| To determine | | CCGAGGAG3′ | TCTCAGCTC3′ | and 72° C. (30″), (30 cycles) |
| the inflamtory | | Seq ID 41 | Seq ID 42 | |
| state | | | | |
| IL-1α | | 5′GTCTCTGAATCAGAAA | 5′CATGTCAAATTTCACT | 530 bp 95° C. (30″) 62° C. (60″), |
| To determine | | TCCTTCTATC3′ | GCTTCATCC3′ | and 72° C. (30″), (30 cycles) |
| the inflamtory | | Seq ID 43 | Seq ID 44 | |
| state | | | | |
| IFNγ | | 5′ATGAAATATACAAGTT | 5′GATGCTCTTCGACTTC | 384 bp 95° C. (30″) 62° C. (60″), |
| To determine | | ATATCTTGGCTTT3′ | GAAACAGCAT3′ | and 72° C. (30″), (30 cycles) |
| the inflamtory | | Seq ID 45 | Seq ID 46 | |
| state | | | | |

Validation of markers is accomplished according to criteria established by the Working Group on Molecular and Biochemical Markers of Alzheimer's Disease and worth to evaluate (i) sensitivity (refers to the capacity of a biomarker to identify a substantial percentage of patients with the disease), (ii) specificity (refers to the capacity of a test to distinguish AD from normal aging, other causes of cognitive disorders and dementias); (iii) predictive (positive or negative) value (represents the percentage of people with a positive/negative test who subsequently at autopsy prove to have/not to have the disease).

### Plasma Lipid Testing

Heparinized plasma specimens are collected for lipid testing after an overnight fast and analyzed on the same day. Total cholesterol (TC), triglycerides (TG) and phospholipid (PL) levels are determined enzymatically (Boehringer Mannheim Diagnostics, Indianapolis, Ind.). High-density lipoprotein cholesterol (HDL-C) are determined after precipitation of the apolipoprotein B (Apo-B) containing particles by magnesium chloride and dextran sulfate.

### Intracellular Lipid Content

For lipid cell content determinations, neutral lipids extracted from isolated cultured PBMCs and skin fibroblasts with cold acetone, are separated by thyn layer chromatography (TLC). Free cholesterol (FC), cholesterol esters (CE), triglycerides (TG) and phospholipids (PH) mass are determined by enzymatic standard assay methods.

### Neutral Lipid Staining

For neutral lipid staining, isolated PBMC and skin fibroblasts are cultured as described above. At different times of incubation, the cells are washed three times with PBS and fixed by soaking in 10% formalin. The cells are treated with isopropylic alcohol (60%), washed and nuclei and intracellular neutral lipid droplets are then stained with Mayer's hematoxylin solution and oil red O, respectively. The stained cells are then examined and photographed under the light microscopy. Lipid-bound ORO was quantified in intact cells or in cell extracts by the Scion image analysis software (NIH Image 1.63 Analysis Software program) or after chloroform/methanol (2:1) extraction of lipids and OD reading at 520 nm, respectively.

### Results

### Alteration of Cholesterol Homeostasis in Neoplastic and Atherosclerotic Cells

Table 4 shows lipid content in primary Acute and Chronic Lymphocytic Leukemia (ALL and CLL, respectively) cells, as well as lipid profiles of sera from patients with CLL (18 patients, ages 45-65 years), or ALL (12 patients, ages 40-60 years), at diagnosis. Age-matched healthy subjects (n=15) were used as controls. The authors found that constitutive cholesterol ester levels were higher in leukemia cells than in controls. A strong decrease in FC:CE molar ratio (1.1 in CLL and 0.85 in ALL vs. 3.6 in controls) was also observed in leukemia cells. No significant changes in other cellular lipid parameters were seen. HDL-C were significantly reduced (P<0.05) in leukemia patients compared with age-matched healthy controls. Total serum cholesterol (TC), LDL-C, TG, and PL levels were not significantly different between control subjects and tumor patients, although a trend toward hypocholesterolemia and hypertriglyceridemia was observed in the latter group (data not shown).

TABLE 4
Correlation between intracellular free and esterified cholesterol
and plasma HDL cholesterol in proliferative diseases.

| | FC | CE | HDL-Cholesterol |
|---|---|---|---|
| Proliferative Disease | μg/10⁶ cells | | mg/dl |
| ALL | 1.7 | 2.0 (0.85) | 7-20 |
| CLL | 2.0 | 1.8 (1.1) | 12-27 |
| Controls | 2.5 | 0.7 (3.6) | 40-60 |

Numbers in parenthesis are the ratio FC:CE.

These results support the notion that changes in lipid content, mainly in the levels of cellular cholesterol esters and plasma HDL-C, represent an identifiable profile for proliferative diseases.

To reinforce these results, the authors analyzed the cytoplasm lipid content in normal lymphocytes and in leukemic PBMCs, using the oil-red O staining method.

As shown in FIG. 3A, at time 0 only leukemic PBMCs are positively stained (as indicated by the presence of red spots in cells shown with the arrows), while 24 h and 48 h after PHA stimulation control cells also become positive. The intensity of the staining is proportional to the amount of cholesterol esters. The FIG. 3A shows that lipid accumulation is higher in leukemic cells than in control cells at all time points considered. The results showed are representative of 7 different patients and 7 control samples. Moreover, ACAT-1 mRNA levels were higher while neutral cholesterol ester hydrolase (nCEH) and ABCA1 mRNA levels were lower in PBMCs from leukemic patients compared to healthy controls (FIG. 3 B).

Similar results were obtained in mitogen-induced proliferating PBMCs from atherosclerotic patients compared with mononuclear cells from healthy control subjects (FIG. 4).

### Alteration of Cholesterol Homeostasis in Skin Fibroblasts of AD Patients

In vitro studies on cellular trafficking and metabolism of cholesterol (reported in FIG. 5 and 5Bis) reveal that skin fibroblasts from AD patients have an increased capacity to esterify and accumulate cholesterol when compared to fibroblasts from healthy donors. Moreover, the authors found higher intracellular levels of mRNA for MDR1-Pgp and ACAT-1, with lower levels of mRNA for nCEH, caveolin-1 and ABCA1, in fibroblasts from the same AD patients compared to healthy controls (FIG. 5B is A). This was accompanied by an increase in ACAT and a decrease in caveolin 1 protein levels (FIG. 5B is B). APOE subject aplotypes are shown in the Table of FIG. 5B is.

### Alteration of Cholesterol Homeostasis in PBMCs of AD Patients

The data reported in FIGS. 6 and 7 reveal that alterations in cholesterol esters metabolism and trafficking as described in skin fibroblasts are also present in PBMCs isolated from AD patients and their relatives. In particular, as shown in FIG. 7B, the majority of PBMCs from AD patients (65%) had ORO positivity values that scored between 3 and 4, about 80% of controls had values of 0, while most AD relatives scored between 1 and 2. These data further demonstrate the existence of an unbalance in cholesterol metabolism in cells from AD patients that leads to CE accumulation in the cytoplasm and decreased FC export, as evidenced by the low levels of HDL-C shown in FIG. 7A, and decreased expression levels of nCEH, Cav-1, and ABCA-1 mRNA, as shown in FIG. 8A,B,C.

### Alteration of Cholesterol Homeostasis in PBMCs and Skin Fibroblasts of Scrapie-Infected Sheep

Similar correlations were also found in brain tissue (FIG. 9B), plasma (FIG. 9A) and skin fibroblasts (FIG. 10,11) from scrapie-infected and not infected sheep with scrapie-susceptible genotype ARQ/ARQ (ARQ/ARQ+ and ARQ/ARQ−) as compared to control uninfected animals with scrapie-resistant genotype (ARR/ARR). In fact, cholesterol esterification rates (FIG. 10B) and intracellular CE content (FIG. 11) were significantly higher, while plasma HDL-cholesterol was lower (FIG. 9A) in the scrapie-susceptible genotype sheep compared to the scrapie-resistant genotype animals. Importantly, as found by the authors in other cell systems, increased levels of CE observed in the scrapie-susceptible genotype skin fibroblasts were associated with an increased growth rate (FIG. 10A).

In conclusion, the presence of high intracellular CE levels and, consequently, low plasma membrane FC is indicative of a cell phenotype predisposing to several pathological conditions (FIG. 2A,B,C), including formation/deposition of structurally aberrant proteins in conformational diseases (FIG. 2C). The present results are supported by recent studies that have shown that blocking intracellular CE accumulation by ACAT inhibitors prevents the generation of beta-amyloid peptides in cell-based models of AD (34), and strongly inhibits the formation of cerebral amyloid plaques in a mouse model of AD (35).

In addition, the authors had already shown that some of the above parameters also correlate with the ageing phenomenon (36). In fact, using a rat model, the authors proved that, as the age of animals increases, the levels of MDR1-Pgp and ACAT mRNAs significantly increase in several organs (liver, brain, hearth, kidney, arteries). By contrast, the expression levels of mRNA for nCEH and caveolin-1 significantly decrease with age, leading to intracellular CE accumulation and to decreased levels of plasma membrane FC, coupled with low circulating HDL-cholesterol (36). The present results demonstrate that the above correlations can also be found in PBMCs and skin fibroblasts, thus allowing a prompt evaluation of the risk of developing the above defined age-related diseases.

An exemplificative kit for measuring the amount of cytoplasmic CE accumulation in a pheripheral blood sample may take advantage of the ORO staining method, and may include:

a) means for isolating PBMCs from whole blood samples, i.e. according to the following procedure:
Two ml of whole blood are diluted 1:1 with PBS.
The diluted samples are stratified on 2 ml of Lymphoprep in a 15 ml centrifuge tube,
Tubes are centrifuged for 15 min. at 2200 rpm.
The ring containing mononuclear cells is collected with a Pasteur pipette and washed with RPMI 1640.
Cells are resuspended in 3-5 ml of Hank's Balanced Salt Solution (HBSS) and counted.
   b) means for the detection of cytoplasmic CE by ORO staining, i.e. according to the following procedure:
An aliquot of 1×10⁶ cells is transferred to a round bottom borosilicate tube, fixed with 10% formalin for 30 min and centrifuged at 500 rpm.
The cell pellet is resuspended and stained with 1 ml of ORO (1:200 v/v in isopropyl alcohol) for 10 min at room temperature under continuous agitation.
The excess ORO stain is discarded and the cells are counterstained with Mayer's Hematoxylin.
An aliquot of stained cells (10 μl) is placed on a glass slide and inspected by light microscopy and photographed.
   c) means for the spectrophotometric quantification of lipid-bound ORO i.e. according to the following procedure:
The remaining cells are treated with 3 volumes of a mixture of chloroform/methanol (2:1 v/v) and vortexed to extract cell lipids.
The tube is then centrifuged for 10 min. at 500 rpm at room temperature to separate the chloroformic phase which is collected and transferred to a quartz cuvette.
The optical absorbance of the chloroformic phase is then measured at 520 nm (the maximal optical absorbance of ORO).
Absorbance values are plotted against a standard curve and expressed as mmol of ORO per number of cells.

An exemplificative kit for the relative quantification of at least one mRNA involved in intracellular cholesterol homeostasis may include means for the specific reverse amplification of mRNA through cDNA or fragment thereof.

An exemplificative Polymerase Chain Reaction (PCR) reaction reagent mix includes:
MgCl2 (2 mM)
Target cDNA (10 μL)
Digoxigenin-11-2′-deoxy-uridine-5′-triphosphate (DIG) (8 μM)
primer forward and reverse (included but not limited to the sequences listed in Table 3 ACAT, or MDR1, or nCEH, or caveolin-1, or ABCA1) (0.15 μM) and primer standard β-actin (0.15 μM)
AmpliTaq Gold (Taq polymerase: 1.25 U)
1×PCR BufferII (500 mM KCl; 100 mM TRIS-HCl, pH 8.3)
H₂0 to volume
means for the purification of PCR products

PCR products are loaded onto filtration columns (ex. Microspin™ G-50, Amersham Pharmacia) and washed according to the protocol provided by the manufacturer.

Eluted amplicons are collected in clean tubes, and stored at −20° C. if not used immediately.

### Preparation of Macroarrays:

Macroarrays are prepared by printing purified PCR products, suspended in DR.DiY spotting buffer, using the Fast Spotter macroarrayer. Printing is done on aminosilane-treated slides (ex. CMT-GAPS™ from Corning). After printing, slides are allowed to dry at room temperature and then UV-crosslinked with a UVC 500 crosslinker (Hoefer) and used immediately or stored dessicated at room temperature.

Preparation of Labelled cDNAs (in Each of 2 Separate 0.5 ml Eppendorf Tubes)

| | | |
|---|---|---|
| PolyA RNA (from either the sample or control) | 2 | μg |
| oligo - (dT) primer (18-20mer) 1 μg/μl | 2 | μl |
| DEPC-trated water to 10 μl | X | μl |
| Heat at 70° C. for 10 min and chill on ice | | |

### Preparation of First Strand Reaction Mix

| | | |
|---|---|---|
| 5X first strand buffer | 6 | μl |
| dNTP mix (10 mM each) | 1.5 | μl |
| DTT (0.1M) | 3 | μl |
| RNase out | 1 | μl |
| DEPC-trated water to 30 μl | 18.5 | μl |
| To each tube containing the denatured mRNA are added | | |
| First strand reaction mix | 15 | μl |
| Cy3 or Cy5 dUTP 1 mM | 3 | μl |
| Superscript ™ II reverse transcriptase (200 U/μl) | 2 | μl |

The reactions are mixed, the tubes are wrapped in aluminum foil and incubated at 42° C. for 2 hours.

Tubes are then pulse-spun and 1.5 μl of EDTA are added to stop the reaction

### Labelled Probe Cleaning:

Labeling reactions are loaded onto filtration columns (ex. Microspin™ G-50, Amersham Pharmacia) and washed according to the protocol provided by the manufacturer.

Eluted probes are collected in clean tubes, wrapped in foil, and stored at −20° C. if not used immediately.

### Array Hybridization:

Equal volumes (10 μl) of the two probes are combined in a 0.5 ml Eppendorf tube and then are added:

| | |
|---|---|
| COT1 DNA (20 μg/μl) | 1 μl |
| Poly A RNA (20 μg/μl) | 1 μl |

Probes are denatured by heating at 95° C. for 3 min. and then combined with an equal volume of hybridization buffer (10×SSC, 50% formamide, 0.2% SDS).

Just before use, slides are prehybridized in a Coplin jar with Pre-hyb buffer (5×SSC, 0.1% SDS and 1% BSA) for 30 min at 42° C.

Slides are dipped in filtered Milli-Q water and then in isopropanol and allowed to dry at room temperature.

Thirty μl of probe mix are overlaid onto the macroarray and covered with a 22×60 mm hydrophobic coverslip.

Slides are set in Hybridization chambers which are then sealed with the lid and placed in a water bath at 42° C. and incubated for 16-20 hours in the dark.

After hybridization slides are placed in a Petri dish, submerged in wash buffer (1×SSC, 0.2% SDS) at 42%.

The coverslips are lifted gently and removed while the slides are submerged and these are then washed with stringency buffer (0.1×SSC, 0.2% SDS).

### Analysis and Quantification of Hybridised Macroarrays

After washing the slides are allowed to dry at room T and immediately scanned with a GenePix® 4000B Fluorescent scanner and analyzed with the GenePix Pro software. Relative levels of expression are calculated as red-to-green signal ratio, after normalization and background subtraction.

### REFERENCES

1. Metherall, J. E., Li, H., and Waugh, K. J Biol Chem 1996; 271, 2634-2640
2. Debry, P., Nash, E. A., Neklason, D. W., and Metherall, J. E. J Biol Chem 1997; 272, 1026-1031
3. Luker, G. D., Nilsson, K. R., Covey, D. F., and Piwnica-Worms, D. J Biol Chem 1999; 274, 6979-6991
4. Brown M S, Ho Y K, Goldstein J L. J Biol Chem 1980; 255, 9344-52
5. Lange, Y., Ye, J., and Chin, J. J Biol Chem 1997; 272, 17018-17022
6. Lange, Y., and Steck, T. J Biol Chem 1994; 269, 29371-29374
7. Field, E. J., Born, E., Murthy, S., and Mathur, S. N. J Lipid Res 1998; 39, 1938-1950
8. Smart, E. J., Ying, Y., Donzel, D. C., and Anderson, R. G. J Biol Chem 1996; 271, 29427-29435
9. Fielding, P. E., and Fielding, C. J. Biochemistry 1995; 34, 14288-14292
10. Kurzchalia, T. V., and Parton, R. G. Curr Op Cell Biol 1999; 11, 424-431
11. Simons K and Ehehalt R. J Clin Invest 2002; 110, 597-603.
12. Wang P, Weng J, Anderson R. G. W. Science 2005: 307. 1472-1476
13. Simons M et al. Neurology 2001; 57, 1089-1093.
14. Diomede L, Forloni G, Bugiani O, et al. Neurobiol Lipids 2002; 1, 8-14.
15. Rao K. N., ottapally S. K, Eskander E. D, Shinozuka H., Dessi S., Pani P. Br. J. Cancer 1986; 54: 305-310.
16. S. Dessi, B. Batetta, C. Anchisi, P. Pani, P. Costelli, L. Tessitore, F. M. Baccino. Br. J. Cancer 1992; 66: 787-793.
17. Dessi S., Batetta B., Pulisci D., Accogli P., Pani P., Broccia G. Int. J. Hematol 1991; 54: 483-486.
18. Dessi S., Batetta B., Pulisci D., Spano O., Cherchi R., Lanfranco G., Tessitore L., Costelli P., Baccino F. M., Anchisi C., Pani P. Oncology 1992; 49: 436-441
19. Dessi S., Batetta B., Pulisci D., Spano O., Anchisi C., Tessitore L., Costelli P., Baccino F. M., Aroasio E., Pani P. Cancer 1994; 73: 253-258.
20. Dessi S., Batetta B., Spano O., Sanna F., Tonello M., Giacchino M., Tessitore L., Costelli P., Baccino F. M., Madon E., Pani P. Clin. Sci 1995, 89: 505-510.
21. Pani A, Batetta B., Putzolu M., Sanna F, Spano O., Piras S., Mulas M F., Bonatesta R R, Amat di S. Filippo C., Vargiu L., Marceddu T., Sanna L., La Colla P., Dessi S. Cell Mol Life Science 2000; 57: 1094-1103.
22. Dessi, S., Batetta, B., Pani, A., Spano, O., Sanna, F., Putzolu, M., Bonatesta, R., Piras, S., and Pani, P. Biochem J 1997; 321, 603-608
23. Batetta, B., Pani, A., Putzolu, M., Sanna, F., Bonatesta, R. R., Piras, S., Spano, O., Mulas, M. F., and Dessi, S. Cell Prolif 1999; 32, 49-61
24. Batetta, B., Mulas, M. F., Petruzzo, P., Putzolu, M., Bonatesta, R. R., Sanna, F., Cappai, A., Brotzu, G., and Dessi S. Cell Mol Life Sci 2001; 58, 1113-1120
25. Trzesniewska K., Brzyska, Elbaum D. Acta Neurobiol Exp 2004; 64, 41-52.
26. Kaytor M D and Warren S T. J Biol Chem 1999; 37507-37510.
27. Ferrer I, Blanco R, Carmona M, et al. Acta Neuropathol 2001; 101, 49-56.
28. Selkoe, D. J. (2001). Physiol Rev 81, 741-766
29. Aguzzi A, Heikenwalder M, Miele G. J Clin Invest 2004; 114, 153-160.
30. Harper J D and Lansbury P T. Annu Rev Biochem 1997; 66, 385-407.
31. Churcher I, Beher D. Curr Pharm Des. 2005; 11, 3363-82.
32. Racchi M, Mazzucchelli M, Lenzken S C, Porrello E, Lanni C, Govoni S. Chem Biol Interact. 2005; 157-158:335-338.
33. Pavlov O V, Bobryshev YuV, Balabanov YuV, Ashwell K. Neurotoxicol Teratol. 1995; 17, 31-9
34. Puglielli, L., Konopka, G., Pack-Chung, E., Ingano, L. A., Berezovska, O., Hyman, B. T., Chang, T. Y., Tanzi, R. E., Kovacs, D. M. Nat Cell Biol 2001; 3, 905-912.
35. Hutter-Paler, B. H. et al. Neuron 2004; 44, 237-238.
36. Mulas M. F., Demuro G., Mulas C., Putzolu M., Cavallini G., Donati A., Bergamini E. and Dessi S. Mechanisms of Ageing and Development 2005, 126: 648-54.
37. Nicolosi R J, Wilson T A, Krause B R. Atherosclerosis. 1998; 137(1):77-85.
38. Insull W Jr, Koren M, Davignon J, Sprecher D, Schrott H, Keilson L M, Brown A S, Dujovne C A, Davidson M H, McLain R, Heinonen T. Atherosclerosis. 2001; 157, 137-44.
39. Panyam J, Labhasetwar V. Advanced Drug Delivery Reviews. 2003; 55, 329-47.
40. Péan J M, Menei P, Morel O, Montero-Menei C N, Benoit J P. Biomaterials. 2000; 21, 2097-101.
41. Verdun, P. Couvreur, H. Vranckx, V. Lenaerts, M. Roland. J. Control. Release 1986; 3, 205-210.
42. Muller B., Kreuter J. Int. J. Pharm. 1999; 178, 23-32
43. Butcher J, Brown H. Lancet. 2004; 363, 1201-1208.

1. A method to diagnose and/or to make prognostic predictions and/or to monitor the efficacy of a therapy of a proliferative or conformational disease, or to establish the state of ageing in a subject, comprising the steps of: a) collecting a blood sample from the subject; b) separating plasma and isolating peripheral blood lymphocytes (PBMCs) from the blood sample; c) measuring the level of HDL-cholesterol in the plasma; d) culturing PBMCs and promoting their proliferation by stimulation with an appropriate efficient amount of a stimulating agent to get a sufficient amount of cultured PBMCs; e) isolating the lipid fraction from the cultured PBMCs; f) determining the amount of free and esterified cholesterol from the isolated lipid fraction of cultured PBMCs; g) detecting at least one mRNA, and/or its translated protein involved in intracellular cholesterol homeostasis in the cultured PBMCs; and, optionally h) detecting at least one mRNA and/or its translated protein involved in the pathogenesis of specific proliferative or conformational diseases in the cultured PBMCs; and-, optionally i) detecting at least one mRNA and/or its translated protein of pro-inflammatory cytokines in the cultured PBMCs; j) comparing the results of steps c), f), g), h), i) with standardized values from an age-matched control sample.

2. The method according to claim 1 wherein the proliferative disease is selected from the group of: atherosclerosis, restenosis after angioplastic, hematologic neoplasms, solid tumors.

3. The method according to claim 2 wherein the hematologic neoplasms are selected from the group of: Hodgkin and non-Hodgkin lymphomas, acute and chronic leukemias, eritroleukemias, mielomas or policytemias.

4. The method according to claim 2 wherein the solid tumors are selected from the group of: brain, headneck, nasopharyngeal, breast, lung, gastrointestinal, colon, kidney or liver tumor.

5. The method according to claim 1 wherein the conformational disease is selected from the group of: prion-related disorders, Alzheimer's disease, Parkinson's disease, Huntington disease, amyotrophic lateral sclerosis or spinocerebellar degenerations.

6. The method according to claim 5 wherein the prion-related disorders are selected from the group of: Creutzfeldt-Jacob disease, new variant Creutzfeldt-Jacob disease, Gerstmann-Straussler Sheinker syndrome, fatal familial insomnia, bovine spongiform encephalopathy, scrapie, chronic wasting disease, feline spongiform encephalopathy.

7. The method according to claim 1 wherein the stimulating agent is a mitogenic agent.

8. The method according to claim 7 wherein the mitogenic agent is phytohemagglutinin or Concanavalin A.

9. A method to diagnose and/or to make prognostic predictions and/or to monitor the efficacy of a therapy of a proliferative or conformational disease, or to establish the state of ageing in a subject, comprising the steps of: a) collecting a blood sample from the subject; b) separating plasma and isolating peripheral blood lymphocytes (PBMCs) from the blood sample; c) measuring the level of HDL-cholesterol in the plasma: d) isolating fibroblasts from the subject; e) culturing, synchronizing and optionally stimulating isolated fibroblasts to obtain a sufficient amount of cells; f) isolating lipid fraction from cultured fibroblasts; g) determining the amount of free and esterified cholesterol in the isolated lipid fraction or in the cultured fibroblasts; h) detecting at least one mRNA and/or translated protein involved in intracellular cholesterol homeostasis in the cultured fibroblasts; and, optionally i) detecting at least one mRNA and/or translated protein involved in the pathogenesis of specific proliferative or conformational diseases in the cultured fibroblasts; and, optionally j) detecting at least one mRNA or translated protein of pro-inflammatory cytokines in the cultured fibroblasts; k) comparing the results of steps c), g), h), i) and j) with standardized values from an age-matched control sample.

10. The method according to claim 9 wherein the synchronizing fibroblasts step of e is performed by serum deprivation.

11. The method of claim 9 wherein the stimulating fibroblasts step of e) is performed by addition of fetal calf serum or at least one mitogen.

12. The method of claim 11 wherein the mitogen is β-FGF.

13. The method according to claim 1 wherein the esterified cholesterol is measured by staining of cells with oil red O.

14. The method according to claim 1 wherein the mRNA and/or translated protein involved in intracellular cholesterol homeostasis is comprised in the group of LDL-R, HMGCoA-R, SREBP2, MDR1, ACAT-1, Caveolin-1, nCEH and ABCA1.

15. The method according to claim 1 wherein the mRNA and/or translated protein involved in the pathogenesis of the proliferative and conformational is comprised in the group of: APP, Neprilysin, β-secretase; PrP protein; tumor suppressor genes such as pi 6, p53, PTEN and oncogenes such as cMyc, Cyclin DI, ErbB2, EGF-R and Bcl2.

16. The method according to claim 1 wherein the mRNA and/or translated protein of pro-inflammatory cytokines is selected in the group of: Tumour Necrosis Factor alpha (TNFα), Interleukin-1 alpha (IL-Ia) and Interferon-gamma (IFNγ).

17. The method according to claim 1 further comprising the step of determining the ApoE aplotype.

18-29. (canceled)

30. The method according to claim 9 wherein the esterified cholesterol is measured by staining of cells with oil red O.

31. The method according to claim 9 wherein the mRNA and/or translated protein involved in the pathogenesis of the proliferative and conformational is comprised in the group of: APP, Neprilysin, β-secretase; PrP protein; tumor suppressor genes such as pi 6, p53, PTEN and oncogenes such as cMyc, Cyclin DI, ErbB2, EGF-R and Bcl2.

32. The method according to claim 9 wherein the mRNA and/or translated protein of pro-inflammatory cytokines is selected in the group of: Tumour Necrosis Factor alpha (TNFα), Interleukin-1 alpha (IL-Ia) and Interferon-gamma (IFNγ).

33. The method according to claim 9 further comprising the step of determining the ApoE aplotype.


SEQUENCE LISTING (46 sequences)

SEQ ID NO: 1 - DNA, 20 bp, Artificial
  primer ACAT for
agcagaggcagaggaattga

SEQ ID NO: 2 - DNA, 20 bp, Artificial
  synthetic primer
gcacacctggcaagatggag

SEQ ID NO: 3 - DNA, 20 bp, Artificial
  synthetic primer
cccatcattgcaatagcagg

SEQ ID NO: 4 - DNA, 20 bp, Artificial
  synthetic primer
gttcaaacttctgctcctga

SEQ ID NO: 5 - DNA, 20 bp, Artificial
  synthetic primer
cttgtaaacttgagttggag

SEQ ID NO: 6 - DNA, 20 bp, Artificial
  synthetic primer
gtaggaagtaaccacattca

SEQ ID NO: 7 - DNA, 21 bp, Artificial
  synthetic primer
gagcgagaagcaagtgtacga

SEQ ID NO: 8 - DNA, 21 bp, Artificial
  synthetic primer
acagacggtgtggacgtagat

SEQ ID NO: 9 - DNA, 21 bp, Artificial
  synthetic primer
tcctctcccagagcaaaaagc

SEQ ID NO: 10 - DNA, 21 bp, Artificial
  synthetic primer
ctccacaacacttcacatggt

SEQ ID NO: 11 - DNA, 20 bp, Artificial
  synthetic primer
ataccagaatgcagctacta

SEQ ID NO: 12 - DNA, 20 bp, Artificial
  synthetic primer
atctgtcttgatgatctgag

SEQ ID NO: 13 - DNA, 20 bp, Artificial
  synthetic primer
caatgtctcaccaagctctg

SEQ ID NO: 14 - DNA, 20 bp, Artificial
  synthetic primer
tctgtctcgaggggtagctg

SEQ ID NO: 15 - DNA, 20 bp, Artificial
  synthetic primer
taccatgtcaggggtacgtc

SEQ ID NO: 16 - DNA, 22 bp, Artificial
  synthetic primer
caagcctagagacataatcatc

SEQ ID NO: 17 - DNA, 22 bp, Artificial
  synthetic primer
tcagatccgctcccaggttatg

SEQ ID NO: 18 - DNA, 22 bp, Artificial
  synthetic primer
agagtcagccccaaaagaatgc

SEQ ID NO: 19 - DNA, 20 bp, Artificial
  synthetic primer
ctaccgattctttggagttg

SEQ ID NO: 20 - DNA, 20 bp, Artificial
  synthetic primer
cttctcttgctgagaaccac

SEQ ID NO: 21 - DNA, 20 bp, Artificial
  synthetic primer
tctaaaactaggactggtga

SEQ ID NO: 22 - DNA, 20 bp, Artificial
  synthetic primer
ctttagagacagggactgta

SEQ ID NO: 23 - DNA, 20 bp, Artificial
  synthetic primer
attgtcacctagcagataga

SEQ ID NO: 24 - DNA, 20 bp, Artificial
  synthetic primer
ttgttcagtagctcaagtct

SEQ ID NO: 25 - DNA, 20 bp, Artificial
  synthetic primer
atatgccttcccccactacc

SEQ ID NO: 26 - DNA, 20 bp, Artificial
  synthetic primer
cccctgagcttccctagttc

SEQ ID NO: 27 - DNA, 20 bp, Artificial
  synthetic primer
cagtctacctcccgccataa

SEQ ID NO: 28 - DNA, 20 bp, Artificial
  synthetic primer
ccacaacaaaacaccagtgc

SEQ ID NO: 29 - DNA, 24 bp, Artificial
  synthetic primer
ctactcgaggctcccagacatgac

SEQ ID NO: 30 - DNA, 25 bp, Artificial
  synthetic primer
acgctcgagataaaaaaaaattcag

SEQ ID NO: 31 - DNA, 20 bp, Artificial
  synthetic primer
atgcccctcaacgttagctt

SEQ ID NO: 32 - DNA, 17 bp, Artificial
  synthetic primer
gtgggcagctcgaattt

SEQ ID NO: 33 - DNA, 21 bp, Artificial
  synthetic primer
ctggagcccctgaaaaagagc

SEQ ID NO: 34 - DNA, 21 bp, Artificial
  synthetic primer
ctggagaggaagcgtgtgagg

SEQ ID NO: 35 - DNA, 20 bp, Artificial
  synthetic primer
caaccaagtgaggcaggtcc

SEQ ID NO: 36 - DNA, 20 bp, Artificial
  synthetic primer
agaggctgcggattgtgcga

SEQ ID NO: 37 - DNA, 23 bp, Artificial
  synthetic primer
ggaaaagaaaggaaactacgtgg

SEQ ID NO: 38 - DNA, 20 bp, Artificial
  synthetic primer
agttcccgtgggtctagagg

SEQ ID NO: 39 - DNA, 20 bp, Artificial
  synthetic primer
gtgaactgggggaggattgt

SEQ ID NO: 40 - DNA, 20 bp, Artificial
  synthetic primer
acagttccacaaaggcatcc

SEQ ID NO: 41 - DNA, 24 bp, Artificial
  synthetic primer
cgggacgtggagctggccgaggag

SEQ ID NO: 42 - DNA, 24 bp, Artificial
  synthetic primer
caccagctggttatctctcagctc

SEQ ID NO: 43 - DNA, 26 bp, Artificial
  synthetic primer
gtctctgaatcagaaatccttctatc

SEQ ID NO: 44 - DNA, 25 bp, Artificial
  synthetic primer
catgtcaaatttcactgcttcatcc

SEQ ID NO: 45 - DNA, 29 bp, Artificial
  synthetic primer
atgaaatatacaagttatatcttggcttt

SEQ ID NO: 46 - DNA, 26 bp, Artificial
  synthetic primer
gatgctcttcgacttcgaaacagcat